(19) United States
(12) Patent Application Publication
(10) Pub. No.: US 2014/0351998 A1
(43) Pub. Date: Nov. 27, 2014

(54) ENGINEERING PLANTS FOR EFFICIENT UPTAKE AND UTILIZATION OF UREA TO IMPROVE CROP PRODUCTION

(71) Applicant: PIONEER HI BRED INTERNATIONAL INC, Johnston, IA (US)

(72) Inventors: RAJEEV GUPTA, Johnston, IA (US); Zhenglin Hou, Ankeny, IA (US); Dale F. Loussaert, Clive, IA (US); Bo Shen, Johnston, IA (US); Lawrence Kent Wood, Johnston, IA (US)

(73) Assignee: PIONEER HI BRED INTERNATIONAL INC, Johnston, IA (US)

(21) Appl. No.: 14/083,491

(22) Filed: Nov. 19, 2013

Related U.S. Application Data
(60) Provisional application No. 61/728,365, filed on Nov. 20, 2012.
(60) Provisional application No. 61/775,753, filed on Mar. 11, 2013.

(51) Int. Cl. C12N 15/82; C12N 9/80; C05C 9/00; C07K 14/415; C07K 14/395; A01G 1/00; C07K 14/40; C07K 14/39; C07K 14/405; C07K 14/335; C07K 14/195; C07K 14/35; C07K 14/38; C07K 14/32

(52) U.S. Cl. CPC C12N 15/8261; C07K 14/38; C12N 9/80; C05C 9/00; C07K 14/415; C07K 14/395; C07K 14/32; C07K 14/40; C07K 14/39; C07K 14/405; C07K 14/335; C07K 14/195; C07K 14/35; A01G 1/001
USPC 800/278; 536/23.1; 536/23.2; 71/28; 435/320.1; 800/298; 800/320.1; 800/312; 800/322; 800/306; 800/320.3; 800/314; 800/320.2; 800/320; 435/468; 435/252.33; 435/254.2; 435/348; 435/325; 435/419; 435/412; 435/415; 435/416; 435/411; 536/23.6; 536/23.74; 536/23.7; 47/58.1 R

(57) ABSTRACT

The present invention provides polynucleotides and related polypeptides related to urea uptake. The invention provides genomic sequences for urea transporter, urease and glutamine synthetase genes. Urea transporters, urease and glutamine synthetase are responsible for controlling nitrogen utilization efficiency in plants. Urea transporter, urease or glutamine synthetase sequences are provided for improving grain yield and plant growth. The invention further provides recombinant expression cassettes, host cells and transgenic plants.

### CROSS-REFERENCE TO RELATED APPLICATIONS

This application is a utility application which claims the benefit of U.S. Provisional Patent Application No. 61/728,365, filed Nov. 20, 2012 and U.S. Provisional Patent Application No. 61/775,753 filed Mar. 11, 2013, both of which are hereby incorporated herein in their entirety by reference.

### FIELD

The invention relates generally to the field of molecular biology.

### BACKGROUND

Global demand for nitrogen (N) fertilizer for use in agricultural production currently stands at approximately two hundred million tons, and this figure is expected to triple by 2050 (“Current World Fertilizer Trends and Outlook 2011/12” Food and Agriculture Organization of the United Nations, 2008; Good, et al., 2004 Trends in Plant Science 9:597-605). N fertilizer has typically been applied at economic optimum levels, and this practice has led to a decrease in the percentage of N that is actually absorbed by the crop (Firbank, (2005) Annals of Applied Biology 146:163-175). It is estimated that approximately 60% of the N fertilizer applied to soil is lost through various processes including leaching and surface run-off, denitrification, volatilization, and microbial consumption (Raun and Johnson, (1999) Agronomy Journal 91(3):357-363). Given that the cost of N fertilizer accounts for approximately 20-30% of the total farm operating cost for corn production in the US, the loss of N from the soil represents a significant economic loss. Furthermore the lost N often ends up in rivers and waterways, and this contamination has led to significant environmental impacts including the poisoning of drinking water as well as a dramatic increase in the size of anoxic dead zones in previously rich fishing grounds. Therefore, even a small increase in the efficiency of plant of uptake of N from the soil would lead to significant cost savings to farmers as well as a decrease in the negative environmental effects associated with modern agricultural production methods.

Urea is now the dominant form of N fertilizer applied to agricultural soil. Urea accounts for over forty percent of the total N fertilizer used in the US and significantly more in other parts of the world (Kojima, et al., 2006 J Membr Biol 212:83-91). The extensive use of urea is due to a number of factors including its high N percentage (46.7%), relatively low cost, and ease of storage, transport, and application. However, plants are inefficient at the uptake and use of urea directly as an N source. The majority of urea N applied to the soil is decomposed into ammonium (NH₃) and then converted into nitrate (NO₃) during a soil N cycle defined as nitrification. This process is dependent on the presence of microbes and enzymes in the soil, and often the result is not only the conversion of N into forms that are usable by the plant but also the production of other N intermediates which are lost through volatilization and leaching. For many plants NO₃ is the preferred substrate taken into the roots from the soil, and once inside the cell the NO₃ is reduced to nitrite (NO₂) and then converted once again into NH₃. This NH₃ is finally assimilated into amino acids which can be used by the plant cell, and this entire process of reduction and assimilation is energetically expensive for the developing plant.

While NO₃ is the preferred N source for many plants, it has recently been demonstrated in Arabidopsis and rice that plants possess a specific system for the direct uptake of urea from the environment. In Arabidopsis this system is composed of the Dur3 high affinity transporter (HAT) as well as low affinity transporters (LATs) such as MIPs, NIPs and TIPs. Furthermore, it has long been known that plant cells produce urea endogenously from a number of intracellular processes including the mitochondrial ornithine cycle which metabolizes arginine into urea and ornithine as well as the catabolism of purins or ureides (refs). Urea inside the cell is broken down into NH₃ and CO₂ by a specific enzyme, urease. The NH₃ released is then assimilated into amino acids, primarily through the action of the glutamine synthetase protein (GS).

The existence of plant transport proteins specialized for the uptake of urea is a relatively recent discovery, and it was long believed that urea, a relatively small polar molecule, entered the cell primarily via passive diffusion through the plasma membrane (Lui, et al., 2003 Plant Cell 15:790-800; Galluci, et al., (1971) Arch Int Physiol Biochim 79(5):881-7). However, the discovery in plants of a highly specific active transport system for urea as well as a number of low affinity transporters capable of facilitating passage of urea and other similar molecules has led to renewed interest in the mechanisms of urea homeostasis. These HATs and LATs are thought to be conserved among both dicots and monocots and the ability to transport urea has been shown for both Arabidopsis and Oryza Dur3 homologues. However, relatively little is known about the function and regulation of high affinity urea transporters in other plant species. Most plant genomes encode one presumptive HAT gene which is homologous to the yeast Dur3 gene, the first characterized member of this group of transporters. These genes encode proteins which belong to the sodium-solute symporter family (SSSF), integral membrane proteins which are generally predicted to contain between eleven and fifteen trans-membrane domains (TMD). While many members of the SSSF use the energy of an inwardly directed electrochemical sodium gradient for co-transport of various substrates, it is believed that Dur3 proteins co-transport urea along a proton gradient. These transporters demonstrate saturable kinetics and Kₘ values in the low micromolar range have been calculated using Xenopus expression systems.

In addition to the Dur3 HAT, plants genomes also encode a number of potential low affinity urea transporters. These transporters belong to one of several subfamilies of aquaporins, the tonoplastic intrinsic proteins (TIPs), Nod26-like intrinsic proteins (NIPs) and plasma membrane intrinsic proteins (PIPs). Aquaporins have previously been shown to mediate urea transport in addition to water and other substrates in mammals and research is currently ongoing to determine the exact localization and substrate preferences for this diverse set of proteins in plants. However, examination of a representative family member, the Arabidopsis Tip2-1 gene, shows that encoded protein enhances uptake of urea in substrate concentrations ranging from 100 μM to 30 mM in a pH-independent manner. These relatively low affinity transport kinetics are typically seen in channel type proteins, and this is the predicted mode of urea transport given the structural knowledge of the aquaporin proteins.

The biological necessity of urea transport across cellular membranes is not limited to plants, and various classes of urea transporters have been identified in organisms ranging from bacteria to humans. Perhaps the best studied of these transporters is the Urel-type urea channel of Helicobacter pylori. This integral membrane protein contains six transmembrane domains, and is proton gated. It is believed that Helicobacter transports urea into the cell through Urel and then uses the urease enzyme to break this down into ammonia and carbonic acid. Urea is transported down its concentration gradient as the intracellular urea concentration of urea is relatively low due to high urease activity inside the cell. The breakdown products of urea hydrolysis, ammonia and carbonic acid, are believed to act respectively as extracellular and cytosolic buffers which allow the pathogen to survive in the acidic environment of its host's gastric tract (Sachs, et al., (2006) J Membr Biol 212(2):71-82). However it should be noted that many bacteria use urea hydrolysis as a nitrogen source, and potential Urel genes are encoded in the genomes of bacteria such as Lactobacillus which normally reside in the large intestine of ruminants, a relatively neutral pH environment.

As previously noted urea inside the cell is degraded by urease, a dedicated enzyme which hydrolyzes urea into ammonia and carbonic acid. The urease enzymes are widely distributed in nature and can be found in bacteria, fungi, algae, plants and invertebrates. Beyond their role in intracellular urea homeostasis, it has also been long known that many ureases exist as extracellular enzymes present in the soil. Urease proteins in the soil are responsible for the degradation of urea into ammonia, a practice that is critical to modern urea-fertilization practices. In bacteria, the urease enzyme is a hetero trimer composed of a large, approximately 60 kDa α-subunit and two smaller, 10-20 kDa β- and γ-subunits. Plants and fungi encode these three distinct components in a single polypeptide transcript. These amidohydrolases contain a metal center in their active site, and this active site is typically filled by two nickel [Ni(II)] ions however it can also be composed of other metal ions including iron. This enzyme has been well studied, and in fact it was the first enzyme crystallized by James B. Sumner in 1926 and was the first protein shown to use Ni(II) ions for function.

Once urea has been broken down into its component parts by urease, released ammonia is assimilated into amino acids, primarily through the action of glutamine synthetase (GS) enzymes. To date, three types of GS have been identified in prokaryotes and eukaryotes, GSI, GSII and GSM. Investigation of the published plant genomes reveals that only the cytosolic GSI and plastidic GSII genes are encoded and GSI proteins are encoded by several genes whereas only one gene is known to encode GSII. The GSI genes encode proteins that are believed to be localized to the cytosol and contain both a glutamine synthetase catalytic domain (pfam00120) as well as an amidohydro domain of unknown function (pfam04909) (Forde and Lea, (2007) J Exp Bot 58(9):2339-58; Swarbreck, et al., (2011) J Exp Bot 62(4):1511-22). Little is currently known about the role of specific GSI proteins in cellular processes, however examination of knockout lines for various GSI genes has shown that these plants yield significantly less than wild-type controls in both maize and rice and that overexpression of GS1-3 in maize resulted in a marginal increase in grain yield (Martin, et al., (2006) Plant Cell 18(11):3252-74; Tabuchi, et al., (2007) J Exp Bot 58(9):2319-27). The second type of glutamine synthetase enzyme produced by plants, GSII, is localized primarily in the chloroplast and uses ferredoxin, and thus light energy, as an electron donor and reducing source. GSII is primarily responsible for assimilation of ammonia resulting from nitrate reduction and photorespiration which occurs in vegetative tissues. However, GSII is also expressed in the roots, and it is believed to have a role in assimilation of reduced nitrate in these tissues as well (Swarbreck, et al., 2010 J Exp Bot. 62(4): 1511-22).

The present disclosure includes the identification of a number of putative genes involved in the uptake and assimilation of urea. In particular, approximately two hundred potential urea transporters have been identified and many of these have been tested for the ability to transport urea into the cytosol in cell based assays. As determined from experimental data presented here, “urea-critical” motifs which help to define many of the protein structures required for highly active and specific translocation of urea by Dur3 homologues have also been outlined. While this list of “urea-critical” domains is not exhaustive, future identification of urea transporters using these and other conserved motifs deduced by this method will allow for a greater understanding of the mechanisms of urea movement across cellular membranes. A number of putative urease proteins and glutamine synthetase proteins have also been identified. Approximately 48 potential urease genes have been identified from a variety of sources, principally from sequenced fungal and plant genomes. These genes, which are presumed to function in the breakdown of urea in the cell, are being employed in transgenic stacks in order to create plants that more effectively acquire and utilize urea from the environment, either naturally occurring or applied as a soil or foliar fertilizer supplement.

Transgenic plants expressing either a single or multiple urea transporters or a urea transporter stacked with urease genes for urea breakdown and glutamine synthethase genes for assimilation of urea-derived N into amino acids have also been created. In this way an agricultural system in which urea derived N might be delivered preferentially to plants and thus avoid loss of N into the environment.

### SUMMARY

The present invention provides polynucleotides, related polypeptides and all conservatively modified variants of the urea utilization sequences. The invention provides sequences for the DUR genes. Table 1 lists these genes and their sequence ID numbers.

TABLE 1

| | | | POLYNUCLEOTIDE/ |
|---|---|---|---|
| SEQUENCE ID | IDENTITY | ORGANISM | POLYPEPTIDE |
| SEQ ID NO: 1 | Dur3 | Saccharomyces cerevisiae | Polypeptide |
| SEQ ID NO: 2 | Dur3 | Arabidopsis thaliana | Polypeptide |
| SEQ ID NO: 3 | Dur3 | Zea mays | Polypeptide |
| SEQ ID NO: 4 | gi\|31433710 | Oryza sativa | Polypeptide |
| SEQ ID NO: 5 | gi\|168053949 | Physcomitrella patens | Polypeptide |
| SEQ ID NO: 6 | gi\|302821778 | Selaginella moellendorffii | Polypeptide |
| SEQ ID NO: 7 | gi\|302781086 | Selaginella moellendorffii | Polypeptide |
| SEQ ID NO: 8 | gi\|168065203 | Physcomitrella patens | Polypeptide |
| SEQ ID NO: 9 | gi\|308812023 | Ostreococcus tauri | Polypeptide |
| SEQ ID NO: 10 | gi\|145342521 | Ostreococcus lucimarinus | Polypeptide |
| SEQ ID NO: 11 | gi\|298704872 | Ectocarpus siliculosus | Polypeptide |
| SEQ ID NO: 12 | gi\|255079156 | Micromonas sp. | Polypeptide |
| SEQ ID NO: 13 | gi\|219119631 | Phaeodactylum tricornutum | Polypeptide |
| SEQ ID NO: 14 | gi\|219119635 | Phaeodactylum tricornutum | Polypeptide |
| SEQ ID NO: 15 | gi\|224007933 | Thalassiosira pseudonana | Polypeptide |
| SEQ ID NO: 16 | gi\|302851501 | Volvox carteri | Polypeptide |
| SEQ ID NO: 17 | gi\|159488630 | Chlamydomonas reinhardtii | Polypeptide |
| SEQ ID NO: 18 | gi\|15948863 | Chlamydomonas reinhardtii | Polypeptide |
| SEQ ID NO: 19 | gi\|302851503 | Volvox_carteri | Polypeptide |
| SEQ ID NO: 20 | gi\|288556287 | Bacillus_pseudofirmus | Polypeptide |
| SEQ ID NO: 21 | gi\|294500270 | Bacillus_megaterium | Polypeptide |
| SEQ ID NO: 22 | gi\|169827030 | Lysinibacillus_sphaericus | Polypeptide |
| SEQ ID NO: 23 | gi\|299535887 | Lysinibacillus_fusiformis | Polypeptide |
| SEQ ID NO: 24 | gi\|126649708 | Bacillus sp. | Polypeptide |
| SEQ ID NO: 25 | gi\|194015869 | Bacillus_pumilus | Polypeptide |
| SEQ ID NO: 26 | gi\|229166178 | Bacillus_cereus | Polypeptide |
| SEQ ID NO: 27 | gi\|229084325 | Bacillus_cereus | Polypeptide |
| SEQ ID NO: 28 | gi\|157691433 | Bacillus_pumilus | Polypeptide |
| SEQ ID NO: 29 | gi\|229160294 | Bacillus_cereus | Polypeptide |
| SEQ ID NO: 30 | gi\|229095819 | Bacillus_cereus | Polypeptide |
| SEQ ID NO: 31 | gi\|229084968 | Bacillus_cereus | Polypeptide |
| SEQ ID NO: 32 | gi\|229056972 | Bacillus_cereus | Polypeptide |
| SEQ ID NO: 33 | gi\|229132136 | Bacillus_cereus | Polypeptide |
| SEQ ID NO: 34 | gi\|229016589 | Bacillus_cereus | Polypeptide |
| SEQ ID NO: 35 | gi\|30019384 | Bacillus_cereus | Polypeptide |
| SEQ ID NO: 36 | gi\|295703213 | Bacillus_megaterium | Polypeptide |
| SEQ ID NO: 37 | gi\|294497841 | Bacillus_megaterium | Polypeptide |
| SEQ ID NO: 38 | gi\|163939135 | Bacillus_weihenstephanensis | Polypeptide |
| SEQ ID NO: 39 | gi\|229018434 | Bacillus_cereus | Polypeptide |
| SEQ ID NO: 40 | gi\|228951715 | Bacillus_thuringiensis | Polypeptide |
| SEQ ID NO: 41 | gi\|229010629 | Bacillus_mycoides | Polypeptide |
| SEQ ID NO: 42 | gi\|56420674 | Geobacillus_kaustophilus | Polypeptide |
| SEQ ID NO: 43 | gi\|229189425 | Bacillus_cereus | Polypeptide |
| SEQ ID NO: 44 | gi\|311029285 | Bacillus sp. | Polypeptide |
| SEQ ID NO: 45 | gi\|229154903 | Bacillus_cereus | Polypeptide |
| SEQ ID NO: 46 | gi\|254430694 | Cyanobium sp. | Polypeptide |
| SEQ ID NO: 47 | gi\|254430882 | Cyanobium sp. | Polypeptide |
| SEQ ID NO: 48 | gi\|6321771 | Saccharomyces_cerevisiae | Polypeptide |
| SEQ ID NO: 49 | gi\|50291859 | Candida_glabrata | Polypeptide |
| SEQ ID NO: 50 | gi\|255713520 | Lachancea_thermotolerans | Polypeptide |
| SEQ ID NO: 51 | gi\|50306037 | Kluyveromyces_lactis | Polypeptide |
| SEQ ID NO: 52 | gi\|50545892 | Yarrowia_lipolytica | Polypeptide |
| SEQ ID NO: 53 | gi\|50548823 | Yarrowia_lipolytica | Polypeptide |
| SEQ ID NO: 54 | gi\|320580429 | Pichia_angusta | Polypeptide |
| SEQ ID NO: 55 | gi\|302306588 | Ashbya_gossypii | Polypeptide |
| SEQ ID NO: 56 | gi\|50420461 | Debaryomyces_hansenii | Polypeptide |
| SEQ ID NO: 57 | gi\|260949975 | Clavispora_lusitaniae | Polypeptide |
| SEQ ID NO: 58 | gi\|126274215 | Scheffersomyces_stipitis | Polypeptide |
| SEQ ID NO: 59 | gi\|296414848 | Tuber_melanosporum | Polypeptide |
| SEQ ID NO: 60 | gi\|255730941 | Candida_tropicalis | Polypeptide |
| SEQ ID NO: 61 | gi\|212540098 | Penicillium_marneffei | Polypeptide |
| SEQ ID NO: 62 | gi\|254567651 | Pichia_pastoris | Polypeptide |
| SEQ ID NO: 63 | gi\|189197153 | Pyrenophora_tritici-repentis | Polypeptide |
| SEQ ID NO: 64 | gi\|242802844 | Talaromyces_stipitatus | Polypeptide |
| SEQ ID NO: 65 | gi\|145229001 | Aspergillus_niger | Polypeptide |
| SEQ ID NO: 66 | gi\|255730943 | Candida_tropicalis | Polypeptide |
| SEQ ID NO: 67 | gi\|68484979 | Candida_albicans | Polypeptide |
| SEQ ID NO: 68 | gi\|255941014 | Penicillium_chrysogenum | Polypeptide |
| SEQ ID NO: 69 | gi\|121702817 | Aspergillus_clavatus | Polypeptide |
| SEQ ID NO: 70 | gi\|119496809 | Neosartorya_fischeri | Polypeptide |
| SEQ ID NO: 71 | gi\|67516273 | Aspergillus_nidulans | Polypeptide |
| SEQ ID NO: 72 | gi\|149244852 | Lodderomyces_elongisporus | Polypeptide |
| SEQ ID NO: 73 | gi\|115388603 | Aspergillus_terreus | Polypeptide |
| SEQ ID NO: 74 | gi\|154296456 | Botryotinia_fuckeliana | Polypeptide |
| SEQ ID NO: 75 | gi\|46114538 | Gibberella_zeae | Polypeptide |
| SEQ ID NO: 76 | gi\|302883001 | Nectria_haematococca | Polypeptide |
| SEQ ID NO: 77 | gi\|320589404 | Grosmannia_clavigera | Polypeptide |
| SEQ ID NO: 78 | gi\|85090209 | Neurospora_crassa | Polypeptide |
| SEQ ID NO: 79 | gi\|170940378 | Podospora_anserina | Polypeptide |
| SEQ ID NO: 80 | gi\|310797726 | Glomerella_graminicola | Polypeptide |
| SEQ ID NO: 81 | gi\|19112663 | Schizosaccharomyces_pombe | Polypeptide |
| SEQ ID NO: 82 | gi\|312222180 | Leptosphaeria_maculans | Polypeptide |
| SEQ ID NO: 83 | gi\|289614545 | Sordaria_macrospora | Polypeptide |
| SEQ ID NO: 84 | gi\|169599615 | Phaeosphaeria_nodorum | Polypeptide |
| SEQ ID NO: 85 | gi\|39956134 | Magnaporthe_oryzae | Polypeptide |
| SEQ ID NO: 86 | gi\|213405133 | Schizosaccharomyces_japonicus | Polypeptide |
| SEQ ID NO: 87 | gi\|189192454 | Pyrenophora_tritici-repentis | Polypeptide |
| SEQ ID NO: 88 | gi\|302918614 | Nectria_haematococca | Polypeptide |
| SEQ ID NO: 89 | gi\|322712212 | Metarhizium_anisopliae | Polypeptide |
| SEQ ID NO: 90 | gi\|322697316 | Metarhizium_acridum | Polypeptide |
| SEQ ID NO: 91 | gi\|330934127 | Pyrenophora_teres | Polypeptide |
| SEQ ID NO: 92 | gi\|169604778 | Phaeosphaeria_nodorum | Polypeptide |
| SEQ ID NO: 93 | gi\|330912407 | Pyrenophora_teres | Polypeptide |
| SEQ ID NO: 94 | gi\|259483267 | Aspergillus_nidulans | Polypeptide |
| SEQ ID NO: 95 | gi\|320582983 | Pichia_angusta | Polypeptide |
| SEQ ID NO: 96 | gi\|212539780 | Penicillium_marneffei | Polypeptide |
| SEQ ID NO: 97 | gi\|134111404 | Cryptococcus_neoformans | Polypeptide |
| SEQ ID NO: 98 | gi\|238499135 | Aspergillus_flavu | Polypeptide |
| SEQ ID NO: 99 | gi\|169856863 | Coprinopsis_cinerea | Polypeptide |
| SEQ ID NO: 100 | gi\|255933690 | Penicillium_chrysogenum | Polypeptide |
| SEQ ID NO: 101 | gi\|302887803 | Nectria_haematococca | Polypeptide |
| SEQ ID NO: 102 | gi\|212547032 | Penicillium_marneffei | Polypeptide |
| SEQ ID NO: 103 | gi\|259488035 | Aspergillus_nidulans | Polypeptide |
| SEQ ID NO: 104 | gi\|255946776 | Penicillium_chrysogenum | Polypeptide |
| SEQ ID NO: 105 | gi\|238485376 | Aspergillus_flavus | Polypeptide |
| SEQ ID NO: 106 | gi\|322703973 | Metarhizium_anisopliae | Polypeptide |
| SEQ ID NO: 107 | gi\|67901140 | Aspergillus_nidulans | Polypeptide |
| SEQ ID NO: 108 | gi\|317037515 | Aspergillus_niger | Polypeptide |
| SEQ ID NO: 109 | gi\|302682804 | Schizophyllum_commune | Polypeptide |
| SEQ ID NO: 110 | gi\|71024341 | Ustilago_maydis | Polypeptide |
| SEQ ID NO: 111 | gi\|317033878 | Aspergillus_niger | Polypeptide |
| SEQ ID NO: 112 | gi\|145614668 | Magnaporthe_oryzae | Polypeptide |
| SEQ ID NO: 113 | gi\|115401158 | Aspergillus_terreus | Polypeptide |
| SEQ ID NO: 114 | gi\|156054736 | Sclerotinia_sclerotiorum | Polypeptide |
| SEQ ID NO: 115 | gi\|50312585 | Kluyveromyces_lactis | Polypeptide |
| SEQ ID NO: 116 | gi\|302896644 | Nectria_haematococca | Polypeptide |
| SEQ ID NO: 117 | gi\|119494215 | Neosartorya_fischeri | Polypeptide |
| SEQ ID NO: 118 | gi\|121703898 | Aspergillus_clavatus | Polypeptide |
| SEQ ID NO: 119 | gi\|119467414 | Neosartorya_fischeri | Polypeptide |
| SEQ ID NO: 120 | gi\|328353488 | Pichia_pastoris | Polypeptide |
| SEQ ID NO: 121 | gi\|116194181 | Chaetomium_globosu | Polypeptide |
| SEQ ID NO: 122 | gi\|71020987 | Ustilago_maydis | Polypeptide |
| SEQ ID NO: 123 | gi\|126276093 | Scheffersomyces_stipitis | Polypeptide |
| SEQ ID NO: 124 | gi\|50554215 | Yarrowia_lipolytica | Polypeptide |
| SEQ ID NO: 125 | gi\|68467347 | Candida_albicans | Polypeptide |
| SEQ ID NO: 126 | gi\|170116443 | Laccaria_bicolo | Polypeptide |
| SEQ ID NO: 127 | gi\|150866227 | Scheffersomyces_stipitis | Polypeptide |
| SEQ ID NO: 128 | gi\|238878230 | Candida_albicans | Polypeptide |
| SEQ ID NO: 129 | gi\|254573522 | Pichia_pastoris | Polypeptide |
| SEQ ID NO: 130 | gi\|150864823 | Scheffersomyces_stipitis | Polypeptide |
| SEQ ID NO: 131 | gi\|320589645 | Grosmannia_clavigera | Polypeptide |
| SEQ ID NO: 132 | gi\|121703890 | Aspergillus_clavatus | Polypeptide |
| SEQ ID NO: 133 | gi\|254582434 | Zygosaccharomyce_rouxii | Polypeptide |
| SEQ ID NO: 134 | gi\|115385168 | Aspergillus_terreus | Polypeptide |
| SEQ ID NO: 135 | gi\|50306163 | Kluyveromyces_lactis | Polypeptide |
| SEQ ID NO: 136 | gi\|241953289 | Candida_dubliniensis | Polypeptide |
| SEQ ID NO: 137 | gi\|50554657 | Yarrowia_lipolytica | Polypeptide |
| SEQ ID NO: 138 | gi\|241950425 | Candida_dubliniensis | Polypeptide |
| SEQ ID NO: 139 | gi\|294658033 | Debaryomyces_hansenii | Polypeptide |
| SEQ ID NO: 140 | gi\|255728881 | Candida_tropicalis | Polypeptide |
| SEQ ID NO: 141 | gi\|154302535 | Botryotinia_fuckeliana | Polypeptide |
| SEQ ID NO: 142 | gi\|145247042 | Aspergillus_niger | Polypeptide |
| SEQ ID NO: 143 | gi\|149248528 | Lodderomyces_elongisporus | Polypeptide |
| SEQ ID NO: 144 | gi\|254570247 | Pichia_pastoris | Polypeptide |
| SEQ ID NO: 145 | gi\|83767842 | Aspergillus_oryzae | Polypeptide |
| SEQ ID NO: 146 | gi\|312211148 | Leptosphaeria_maculans | Polypeptide |
| SEQ ID NO: 147 | gi\|310798012 | Glomerella_graminicola | Polypeptide |
| SEQ ID NO: 148 | gi\|70996736 | Aspergillus_fumigatus | Polypeptide |
| SEQ ID NO: 149 | gi\|6752428 | Aspergillus_nidulans | Polypeptide |
| SEQ ID NO: 150 | gi\|242773930 | Talaromyces_stipitatus | Polypeptide |
| SEQ ID NO: 151 | gi\|85711289 | Idiomarina_baltica | Polypeptide |
| SEQ ID NO: 152 | gi\|85712112 | Idiomarina_baltica | Polypeptide |
| SEQ ID NO: 153 | gi\|85713181 | Idiomarina_baltica | Polypeptide |
| SEQ ID NO: 154 | gi\|56460634 | Idiomarina_loihiensis | Polypeptide |
| SEQ ID NO: 155 | gi\|56460286 | Idiomarina_loihiensis | Polypeptide |
| SEQ ID NO: 156 | gi\|56459847 | Idiomarina_loihiensis | Polypeptide |
| SEQ ID NO: 157 | gi\|145220852 | Mycobacterium_gilvum | Polypeptide |
| SEQ ID NO: 158 | gi\|126436855 | Mycobacterium sp. | Polypeptide |
| SEQ ID NO: 159 | gi\|108801019 | Mycobacterium sp. | Polypeptide |
| SEQ ID NO: 160 | gi\|118468997 | Mycobacterium_smegmatis | Polypeptide |
| SEQ ID NO: 161 | gi\|118468841 | Mycobacterium_smegmatis | Polypeptide |
| SEQ ID NO: 162 | gi\|120401830 | Mycobacterium_vanbaalenii | Polypeptide |
| SEQ ID NO: 163 | gi\|145220699 | Mycobacterium_gilvum | Polypeptide |
| SEQ ID NO: 164 | gi\|315442349 | Mycobacterium | Polypeptide |
| SEQ ID NO: 165 | gi\|334140505 | Novosphingobium | Polypeptide |
| SEQ ID NO: 166 | gi\|32477973 | Rhodopirellula_baltica | Polypeptide |
| SEQ ID NO: 167 | gi\|327538200 | Rhodopirellula_baltica | Polypeptide |
| SEQ ID NO: 168 | gi\|32474861 | Rhodopirellula_baltica | Polypeptide |
| SEQ ID NO: 169 | gi\|327538129 | Rhodopirellula_baltica | Polypeptide |
| SEQ ID NO: 170 | gi\|327537996 | Rhodopirellula_baltica | Polypeptide |
| SEQ ID NO: 171 | gi\|32475384 | Rhodopirellula_baltica | Polypeptide |
| SEQ ID NO: 172 | gi\|327537309 | Rhodopirellula_baltica | Polypeptide |
| SEQ ID NO: 173 | gi\|32475554 | Rhodopirellula_baltica | Polypeptide |
| SEQ ID NO: 174 | gi\|32473180 | Rhodopirellula_baltica | Polypeptide |
| SEQ ID NO: 175 | gi\|327541778 | Rhodopirellula_baltica | Polypeptide |
| SEQ ID NO: 176 | gi\|116074213 | Synechococcus | Polypeptide |
| SEQ ID NO: 177 | gi\|78185830 | Synechococcus | Polypeptide |
| SEQ ID NO: 178 | gi\|148242226 | Synechococcus | Polypeptide |
| SEQ ID NO: 179 | gi\|318041913 | Synechococcus | Polypeptide |
| SEQ ID NO: 180 | gi\|33866985 | Synechococcus | Polypeptide |
| SEQ ID NO: 181 | gi\|88809430 | Synechococcus | Polypeptide |
| SEQ ID NO: 182 | gi\|260434524 | Synechococcus | Polypeptide |
| SEQ ID NO: 183 | gi\|78214136 | Synechococcus | Polypeptide |
| SEQ ID NO: 184 | gi\|90655563 | Synechococcus | Polypeptide |
| SEQ ID NO: 185 | gi\|90655390 | Synechococcus | Polypeptide |
| SEQ ID NO: 186 | gi\|87124111 | Synechococcus | Polypeptide |
| SEQ ID NO: 187 | gi\|317969309 | Synechococcus | Polypeptide |
| SEQ ID NO: 188 | gi\|15611137 | Helicobacter_pylori | Polypeptide |
| SEQ ID NO: 189 | gi\|53729263 | Actinobacillus_pleuropneumoniae | Polypeptide |
| SEQ ID NO: 190 | gi\|55820375 | Streptococcus_thermophilus | Polypeptide |
| SEQ ID NO: 191 | gi\|237747731 | Oxalobacter_formigenes | Polypeptide |
| SEQ ID NO: 192 | gi\|335997137 | Lactobacillus_ruminis | Polypeptide |
| SEQ ID NO: 193 | gi\|323703445 | Desulfotomaculum_nigrificans | Polypeptide |
| SEQ ID NO: 194 | ZmTIP4-4 | Zea_mays | Polypeptide |
| SEQ ID NO: 195 | ZmNIP2-4 | Zea_mays | Polypeptide |
| SEQ ID NO: 196 | ZmNIP2-1 | Zea_mays | Polypeptide |
| SEQ ID NO: 197 | AtTIP1-1 | Arabidopsis_thaliana | Polypeptide |
| SEQ ID NO: 198 | AtTIP1-2 | Arabidopsis_thaliana | Polypeptide |
| SEQ ID NO: 199 | AtTIP2-1 | Arabidopsis_thaliana | Polypeptide |
| SEQ ID NO: 200 | AtTIP4-1 | Arabidopsis_thaliana | Polypeptide |
| SEQ ID NO: 201 | NtTIPa | Nicotiana_tabacum | Polypeptide |
| SEQ ID NO: 202 | BdTIP4-4 | Brachypodium_distachyon | Polypeptide |
| SEQ ID NO: 203 | BnTIP4-4 | Brassica_napus | Polypeptide |
| SEQ ID NO: 204 | BrTIP4-4 | Brassica_rapa | Polypeptide |
| SEQ ID NO: 205 | GnTIP4-4 | Glycine_max | Polypeptide |
| SEQ ID NO: 206 | LjTIP4-4 | Lotus_japonicus | Polypeptide |
| SEQ ID NO: 207 | OsTIP4-4 | Oryza_sativa | Polypeptide |
| SEQ ID NO: 208 | PpTIP4-4 | Physcomitrella_patens | Polypeptide |
| SEQ ID NO: 209 | PtTIP4-4 | Populus trichocarpa | Polypeptide |
| SEQ ID NO: 210 | SmTIP4-4 | Selaginella_moellendorffii | Polypeptide |
| SEQ ID NO: 211 | SiTIP4-4 | Setaria_italica | Polypeptide |
| SEQ ID NO: 212 | SbTIP4-4 | Sorghum_bicolor | Polypeptide |
| SEQ ID NO: 213 | VvTIP4-4 | Vitis_vinifera | Polypeptide |
| SEQ ID NO: 214 | BdNIP2-4 | Brachypodium_distachyon | Polypeptide |
| SEQ ID NO: 215 | BrNIP2-4 | Brassica_rapa | Polypeptide |
| SEQ ID NO: 216 | GmNIP2-4 | Glycine_max | Polypeptide |
| SEQ ID NO: 217 | LjNIP2-4 | Lotus_japonicus | Polypeptide |
| SEQ ID NO: 218 | MtNIP2-4 | Medicago_truncatula | Polypeptide |
| SEQ ID NO: 219 | OsNIP2-4 | Oryza_sativa | Polypeptide |
| SEQ ID NO: 220 | PpNIP2-4 | Physcomitrella patens | Polypeptide |
| SEQ ID NO: 221 | PtNIP2-4 | Populus_trichocarpa | Polypeptide |
| SEQ ID NO: 222 | SmNIP2-4 | Selaginella_moellendorffii | Polypeptide |
| SEQ ID NO: 223 | SiNIP2-4 | Setaria_italica | Polypeptide |
| SEQ ID NO: 224 | gi\|302781085_ReORF | Selaginella_moellendorffii | Polynucleotide |
| SEQ ID NO: 225 | gi\|308812022_ReORF | Ostreococcus_tauri | Polynucleotide |
| SEQ ID NO: 226 | gi\|255079155_ReORF | Micromonas | Polynucleotide |
| SEQ ID NO: 227 | gi\|219119634_ReORF | Phaeodactylum_tricornutum | Polynucleotide |
| SEQ ID NO: 228 | gi\|159488629_ReORF | Chlamydomonas_reinhardtii | Polynucleotide |
| SEQ ID NO: 229 | gi\|228951658_ReORF | Bacillus_thuringiensis | Polynucleotide |
| SEQ ID NO: 230 | gi\|56418535_ReORF | Geobacillus_kaustophilus | Polynucleotide |
| SEQ ID NO: 231 | gi\|320580020_ReORF | Pichia_angusta | Polynucleotide |
| SEQ ID NO: 232 | gi\|50423530_ReORF | Debaryomyces_hansenii | Polynucleotide |
| SEQ ID NO: 233 | gi\|118467340_ReORF | Mycobacterium_smegmatis | Polynucleotide |
| SEQ ID NO: 234 | gi\|85711242_ReORF | Idiomarina_baltica | Polynucleotide |
| SEQ ID NO: 235 | gi\|224007932_ReORF | Thalassiosira_pseudonana | Polynucleotide |
| SEQ ID NO: 236 | gi\|316994385_ReORF | Bacillus_pseudofirmus | Polynucleotide |
| SEQ ID NO: 237 | gi\|88809298_ReORF | Synechococcus | Polynucleotide |
| SEQ ID NO: 238 | gi\|168053948_ReORF | Physcomitrella_patens | Polynucleotide |
| SEQ ID NO: 239 | gi\|255713519_ReORF | Lachancea_thermotolerans | Polynucleotide |
| SEQ ID NO: 240 | gi\|302306587_ReORF | Ashbya_gossypii | Polynucleotide |
| SEQ ID NO: 241 | gi\|296414847_ReORF | Tuber_melanosporum | Polynucleotide |
| SEQ ID NO: 242 | gi\|255941013_ReORF | Penicillium_chrysogenum | Polynucleotide |
| SEQ ID NO: 243 | gi\|347976380_ReORF | Podospora_anserina | Polynucleotide |
| SEQ ID NO: 244 | gi\|115483685_ReORF | Oryza_sativa | Polynucleotide |
| SEQ ID NO: 245 | gi\|15611137_ReORF | Helicobacter_pylori | Polynucleotide |
| SEQ ID NO: 246 | gi\|53729263_ReORF | Actinobacillus_pleuropneumoniae | Polynucleotide |
| SEQ ID NO: 247 | gi\|55820103_ReORF | Streptococcus_thermophilus | Polynucleotide |
| SEQ ID NO: 248 | gi\|237747731_ReORF | Oxalobacter_formigenes | Polynucleotide |
| SEQ ID NO: 249 | gi\|335997047_ReORF | Lactobacillus_ruminis | Polynucleotide |
| SEQ ID NO: 250 | gi\|323703406_ReORF | Desulfotomaculum_nigrificans | Polynucleotide |
| SEQ ID NO: 251 | gi\|6319685 | Saccharomyces_cerevisiae | Polypeptide |
| SEQ ID NO: 252 | gi\|343768210 | Naumovozyma_dairenensis | Polypeptide |
| SEQ ID NO: 253 | gi\|254584038 | Zygosaccharomyces_rouxii | Polypeptide |
| SEQ ID NO: 254 | gi\|50308629 | Kluyveromyces_lactis | Polypeptide |
| SEQ ID NO: 255 | gi\|255715439 | Lachancea_thermotolerans | Polypeptide |
| SEQ ID NO: 256 | gi\|50294352 | Candida_glabrata | Polypeptide |
| SEQ ID NO: 257 | gi\|254572936 | Komagataella_pastoris | Polypeptide |
| SEQ ID NO: 258 | gi\|150951195 | Scheffersomyces_stipitis4 | Polypeptide |
| SEQ ID NO: 259 | gi\|5731944 | Schizosaccharomyces_pombe | Polypeptide |
| SEQ ID NO: 260 | gi\|310789956 | Glomerella_graminicola | Polypeptide |
| SEQ ID NO: 261 | gi\|85116050 | Neurospora_crassa | Polypeptide |
| SEQ ID NO: 262 | gi\|296818321 | Arthroderma_otae | Polypeptide |
| SEQ ID NO: 263 | gi\|50788080 | Aspergillus_fumigatus | Polypeptide |
| SEQ ID NO: 264 | gi\|340517340 | Trichoderma_reesei | Polypeptide |
| SEQ ID NO: 265 | gi\|212540166 | Penicillium_marneffei | Polypeptide |
| SEQ ID NO: 266 | gi\|258565811 | Uncinocarpus_reesii | Polypeptide |
| SEQ ID NO: 267 | gi\|295673098 | Paracoccidioides_brasiliensis | Polypeptide |
| SEQ ID NO: 268 | gi\|119496869 | Neosartorya_fischeri | Polypeptide |
| SEQ ID NO: 269 | gi\|303322831 | Coccidioides_posadasii | Polypeptide |
| SEQ ID NO: 270 | gi\|302652330\| | Trichophyton_verrucosum | Polypeptide |
| SEQ ID NO: 271 | gi\|302927660 | Nectria_haematococca | Polypeptide |
| SEQ ID NO: 272 | gi\|317025294 | Aspergillus_niger | Polypeptide |
| SEQ ID NO: 273 | gi\|171687653 | Podospora_anserina | Polypeptide |
| SEQ ID NO: 274 | gi\|326474616 | Trichophyton_tonsurans | Polypeptide |
| SEQ ID NO: 275 | gi\|336267190 | Sordaria_macrospora | Polypeptide |
| SEQ ID NO: 276 | gi\|46107714 | Gibberella_zeae | Polypeptide |
| SEQ ID NO: 277 | gi\|330935671 | Pyrenophora_teres | Polypeptide |
| SEQ ID NO: 278 | gi\|347827989 | Botryotinia_fuckeliana | Polypeptide |
| SEQ ID NO: 279 | gi\|169771067 | Aspergillus_oryzae | Polypeptide |
| SEQ ID NO: 280 | gi\|67516299 | Aspergillus_nidulans | Polypeptide |
| SEQ ID NO: 281 | gi\|238486420 | Aspergillus_flavus | Polypeptide |
| SEQ ID NO: 282 | gi\|108859293 | Agaricus_bisporus | Polypeptide |
| SEQ ID NO: 283 | gi\|301344555 | Oryza_sativa | Polypeptide |
| SEQ ID NO: 284 | gi\|170098735 | Laccaria_bicolor | Polypeptide |
| SEQ ID NO: 285 | gi\|242042716 | Sorghum_bicolor | Polypeptide |
| SEQ ID NO: 286 | gi\|302774665 | Selaginella_moellendorffii | Polypeptide |
| SEQ ID NO: 287 | gi\|353227226 | Piriformospora_indica | Polypeptide |
| SEQ ID NO: 288 | gi\|168056442 | Physcomitrella_patens | Polypeptide |
| SEQ ID NO: 289 | gi\|71023925 | Ustilago_maydis | Polypeptide |
| SEQ ID NO: 290 | gi\|351722260 | Glycine_max | Polypeptide |
| SEQ ID NO: 291 | gb\|M65260.1 | Canavalia_ensiformis | Polypeptide |
| SEQ ID NO: 292 | gi\|225425839 | Vitis_vinifera | Polypeptide |
| SEQ ID NO: 293 | gi\|222143559 | Morus_alba | Polypeptide |
| SEQ ID NO: 294 | gi\|14599436 | Solanum_tuberosum | Polypeptide |
| SEQ ID NO: 295 | gi\|14599434 | Solanum_tuberosum | Polypeptide |
| SEQ ID NO: 296 | gi\|145337258 | Arabidopsis_thaliana | Polypeptide |
| SEQ ID NO: 297 | gi\|297838494 | Arabidopsis_lyrata | Polypeptide |
| SEQ ID NO: 298 | gi\|357123667 | Brachypodium_distachyon | Polypeptide |
| SEQ ID NO: 299 | AtGS1-3 | Arabidopsis_thaliana | Polypeptide |
| SEQ ID NO: 300 | AtGS1-3CDS | Arabidopsis_thaliana | Polynucleotide |
| SEQ ID NO: 301 | ZmGS1-3PRT | Zea_mays | Polypeptide |
| SEQ ID NO: 302 | ZmGS1-3CDS | Zea_mays | Polynucleotide |
| SEQ ID NO: 303 | AtDUR3_Promoter | Arabidopsis thaliana | Polynucleotide |
| SEQ ID NO: 304 | 35S_Promoter | artificial sequence | Polynucleotide |
| SEQ ID NO: 305 | ZmPEPC_Promoter | Zea mays | Polynucleotide |
| SEQ ID NO: 306 | ZmRM2_Promoter | Zea mays | Polynucleotide |
| SEQ ID NO: 307 | AtTUB_Promoter | Arabidopsis thaliana | Polynucleotide |
| SEQ ID NO: 308 | ZmUBI_Promoter | Zea mays | Polynucleotide |
| SEQ ID NO: 309 | RCC3_Promoter | artificial sequence | Polynucleotide |
| SEQ ID NO: 310 | Block_1 | artificial sequence | Polypeptide |
| SEQ ID NO: 311 | Block_2 | artificial sequence | Polypeptide |
| SEQ ID NO: 312 | Block_3 | artificial sequence | Polypeptide |
| SEQ ID NO: 313 | Block_4 | artificial sequence | Polypeptide |
| SEQ ID NO: 314 | Block_5 | artificial sequence | Polypeptide |
| SEQ ID NO: 315 | Urea_critical_motif_1 | artificial sequence | Polypeptide |
| SEQ ID NO: 316 | Urea_critical_motif_2 | artificial sequence | Polypeptide |
| SEQ ID NO: 317 | Urea_critical_motif_3 | artificial sequence | Polypeptide |
| SEQ ID NO: 318 | Urea_critical_motif_4 | artificial sequence | Polypeptide |
| SEQ ID NO: 319 | Urea_critical_motif_5 | artificial sequence | Polypeptide |
| SEQ ID NO: 320 | Urea_critical_motif_6 | artificial sequence | Polypeptide |
| SEQ ID NO: 321 | ZmUreaseCDS | Zea mays | Polynucleotide |
| SEQ ID NO: 322 | ZmUreasePRT | Zea mays | Polypeptide |

Therefore, in one aspect, the present invention relates to an isolated nucleic acid comprising an isolated polynucleotide sequence encoding a urea transporter, a urease or a glutamine synthetase polypeptide. One embodiment of the invention is an isolated polynucleotide comprising a nucleotide sequence selected from the group consisting of: (a) the nucleotide sequence comprising SEQ ID NOS: 224-250, 300, 302 and 321; (b) the nucleotide sequence encoding an amino acid sequence comprising SEQ ID NOS: 1-223, 251-299, 301 and 322 and (c) the nucleotide sequence comprising at least 70% sequence identity to SEQ ID NOS: 224-250, 300, 302 and 321, wherein said polynucleotide encodes a polypeptide having urea transport, urea breakdown, or ammonia assimilation activity.

Compositions of the invention include an isolated polypeptide comprising an amino acid sequence selected from the group consisting of: (a) the amino acid sequence comprising SEQ ID NOS: 1-223, 251-299, 301 and 322 and (b) the amino acid sequence comprising at least 70% sequence identity to SEQ ID NOS: 1-223, 251-299, 301 and 322, wherein said polypeptide has urea transport, urea breakdown or ammonia assimilation activity.

In another aspect, the present invention relates to a recombinant expression cassette comprising a nucleic acid as described. Additionally, the present invention relates to a vector containing the recombinant expression cassette. Further, the vector containing the recombinant expression cassette can facilitate the transcription and translation of the nucleic acid in a host cell. The present invention also relates to the host cells able to express the polynucleotide of the present invention. A number of host cells could be used, such as but not limited to, microbial, mammalian, plant or insect.

In yet another embodiment, the present invention is directed to a transgenic plant or plant cells, containing the nucleic acids of the present invention. Preferred plants containing the polynucleotides of the present invention include but are not limited to maize, soybean, sunflower, sorghum, canola, wheat, alfalfa, cotton, rice, barley, tomato, switchgrass, myscanthus, triticale and millet. In another embodiment, the transgenic plant is a maize plant or plant cells. Another embodiment is the transgenic seeds from the transgenic plant. Another embodiment of the invention includes plants comprising a urea transporter, a urease, or a glutamine synthetase polypeptide of the invention operably linked to a promoter that drives expression in the plant. The plants of the invention can have altered nitrogen use efficiency as compared to a control plant. In some plants, the nitrogen use is altered in a vegetative tissue, a reproductive tissue, or a vegetative tissue and a reproductive tissue. Plants of the invention can have at least one of the following phenotypes including but not limited to: increased leaf size, increased ear size, increased seed size, increased endosperm size, alterations in the relative size of embryos and endosperms leading to changes in the relative levels of protein, oil and/or starch in the seeds, absence of tassels, absence of functional pollen bearing tassels or increased plant size.

Another embodiment of the invention would be plants that have been genetically modified at a genomic locus, wherein the genomic locus encodes a urea transporter, a urease or a glutamine synthetase polypeptide of the invention.

Methods for increasing the activity of a urea transporter, a urease or a glutamine synthetase polypeptide in a plant are provided. The method can comprise introducing into the plant a urea transporter, a urease or a glutamine synthetase polynucleotide of the invention. Providing the polypeptide can decrease the number of cells in plant tissue, modulating the tissue growth and size.

Compositions further include plants and seed having a DNA construct comprising a nucleotide sequence of interest operably linked to a promoter of the current invention. In specific embodiments, the DNA construct is stably integrated into the genome of the plant. The method comprises introducing into a plant a nucleotide sequence of interest operably linked to a promoter of the invention.

### BRIEF DESCRIPTION OF THE FIGURES

FIG. 1: The majority of urea N applied to the soil is decomposed into ammonium (NH₃) and then converted into nitrate (NO₃) during a soil N cycle defined as nitrification. This process is dependent on the presence of microbes and enzymes in the soil, and often the result is not only the conversion of N into forms that are usable by the plant but also the production of other N intermediates which are lost through volatilization and leaching. For many plants NO₃ is the preferred substrate taken into the roots from the soil, and once inside the cell the NO₃ is reduced to nitrite (NO₂) and then converted once again into NH₃. This NH₃ is finally assimilated into amino acids which can be used by the plant cell, and this entire process of reduction and assimilation is energetically expensive for the developing plant. Creation of a transgenic plant capable of efficiently taking up urea directly from the soil will prevent loss of N during microbial conversion processes as well as bypass the necessity to reduce nitrate to ammonia inside the plant cell.

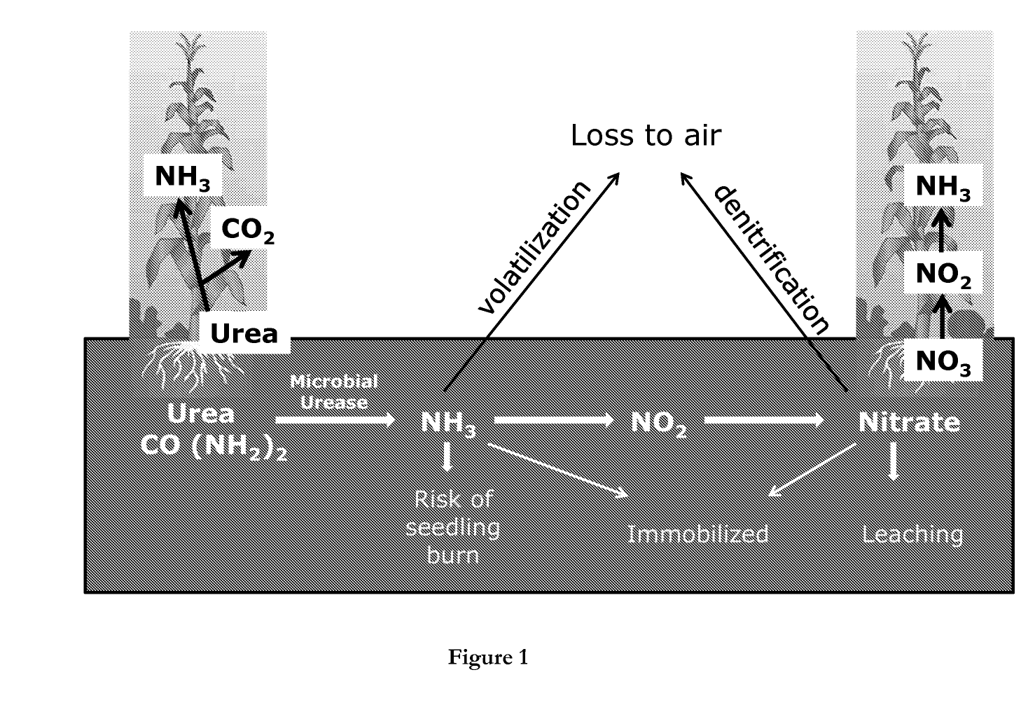

FIG. 2: Urea from the extracellular environment is taken into the cell through specific high-affinity urea transporters such as Dur3, or through less specific low affinity channels such as the aquaporin related NIP proteins. It is speculated that a related group of vacuolar localized proteins, TIPs, function to mobilize urea from intracellular stores. Urea is also produced by endogenous processes such as protein and nucleic acid degradation. Inside the cell urea is degraded by urease, a dedicated enzyme which hydrolyzes urea into ammonia and carbonic acid. Once urea has been broken down into its component parts by urease, released ammonia is assimilated into amino acids, primarily through the action of glutamine synthetase (GS) enzymes but possibly through the action of additional NH₃ metabolizing enzymes.

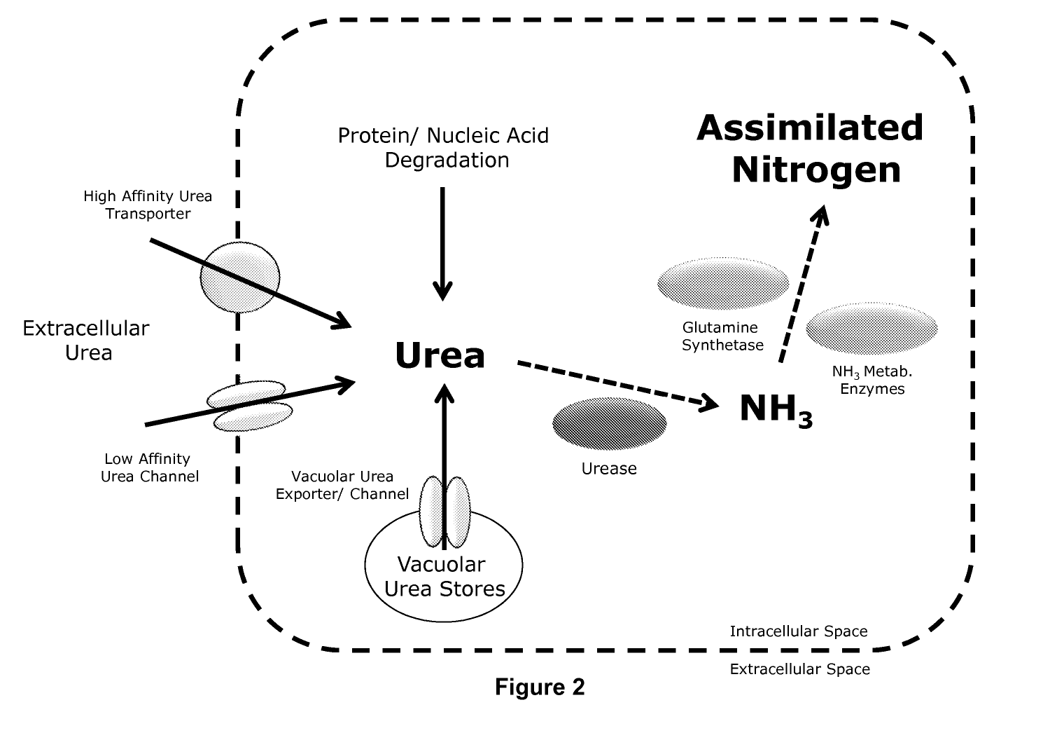

FIG. 3: Sequences of potential DUR3 homologues were obtained from a variety of sources, principally focused on microbes, fungi and lower photosynthetic plants and phylogenetic analysis was performed to examine the relationship among transporters. Proteins which have been confirmed as capable of transporting urea in previous research are shown in green. Transporters from diverse clades which were selected for gene synthesis and subsequent functional characterization are shown in blue.

FIG. 4: B73 maize seedlings were hydroponically grown in a complete synthetic nutrient mix containing 5 mM NO₃ as a nitrogen source until approximately the V4 growth stage. Plants were then either switched to a medium containing no nitrogen or allowed to remain in 5 mM NO₃ (labeled NO₃ in graph). After 3 days growth in no nitrogen media, plants were switched to a growth medium containing 5 mM urea for the 1 hour, 3 hours, 6 hours or 24 hours. Plants were collected and tissues harvested at each of the time points as well as before induction with urea (No nitrogen). qPCR analysis of root tissue from these maize seedlings shows the ZmDur3 is induced by switching to growth media containing 5 mM urea as the sole nitrogen source when compared to plants grown in either no nitrogen or grown in constant amounts of nitrate (FIG. 2).

FIG. 5: A number of genes encoding potential urease proteins have been identified (SEQ ID NOS: 251-298). Forty-eight known or putative ureases were obtained from a variety of sources, primarily of fungal or plant origin. Phylogenetic analysis was performed to examine the relationship among the known and putative urease proteins and these results are presented in the cladogram in FIG. 3.

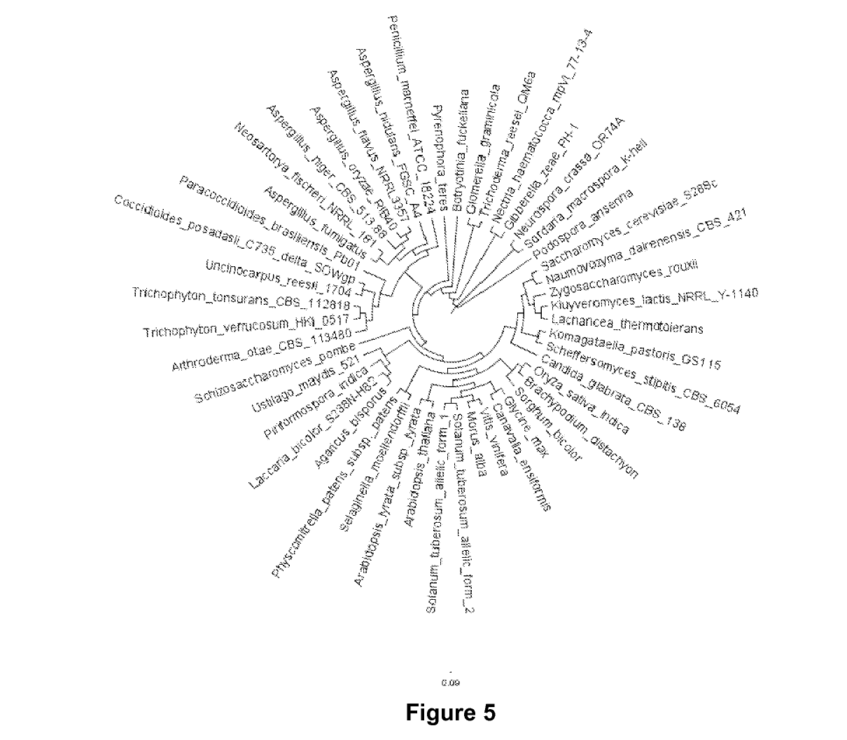

FIG. 6: Protein sequences of DUR3 homologues from Arabidopsis thaliana, Aspergillus nidulans, Debaromyces hansenii, Oryza sativa-Japonica, Phaeodactylum tricornutum, Pichia angusta, Saccharomyces cerevisiae, Selaginella moellendorffii and Synechococcus sp.WH7805 were used to create alignments and identify conserved blocks. These blocks were modeled on alignment files created using the ClustalW2 multiple sequence alignment program and amino acid residues that were absolutely conserved among all of the sequences initially used to define the blocks were identified. Using these conserved residues as a template, a second in silico screen to identify the subset of amino which are not conserved in either the Geobacillus kaustophilus or the Mycobacterium smegmatis sequence (both demonstrate little or no ability to transport urea in a complementation assay) was performed. The residues absolutely conserved in verified transporters and not conserved in either of two non-functional urea transporters are shown in red and these were used to create “urea-critical” motifs which are predicted to have a major positive influence on the ability to transport urea in vivo.

FIG. 7: A 3 dimensional structure topology of the urea transporter core domain and the Rocket-switch mechanism. (a) and (b) A schematic 3D graph of a urea transporter core domain based sodium/galactose transporter from Vibrio parahaemolyticus (vSGLT, PDB:2xq2). The N-terminal half 5 helices are represented by black cylinders while the C-terminal are grey cylinders. In order to delineate the key helical topology clearly, many interhelical elements are omitted. The helix TM1 and its symmetrical counterpart TM6 are unwound at middle. These coiled residues in combination with the middle sections of TM3 and TM8 make up the urea binding site. The model represents an outward-facing conformation. (a) is a view from extracellular side while (b) is viewed along with the pseudo-two-fold rotation axis parallel to the bilayer. (c) A cartoon representation of a rocket-switch or alternative-access mechanism. Although the sequence is intertwined, its 3D structure could be viewed as two subunits A and B. The inter-subunit rotation driven by proton binding enables conformational recycling.

FIG. 8: Sequence/structure alignment and TM helix prediction, were used to identify the 10 core transmembrane helices and the putative urea binding sequence motifs. The first 5 TM domains which compose one half of the symmetrical core protein are shown in the aligment as grey boxes. The 3D model reveals that the putative binding site is at the center of protein, suggesting protein motifs in the middle of sections of TM1, TM3, TM6 and TM8 are important for urea recognition while TM2, TM4, TM5, TM7, TM9 and TM10 likely play a structural role. The residues mutated to test this model are also shown in the figure as dashed ovals. The I180 residue is located in the middle of TM4 and likely interacts with the plasma membrane. Among verified Dur3-type urea transporters this position is conserved and occupied by hydrophobic residues such as I, L and V. Mutation of I180 to aspartic acid (D) introduces a hydrophilic charged residue which should drastically disrupt the structural integrity of the transporter. W72 is near the unwound area of TM1 and is likely involved in urea binding. Both mutated residues fall in or near previously described conserved Blocks 1 and 2 in the N-terminal half of the urea transporter (represented by solid or dashed rectangles, respectively).

FIG. 9: Uptake assays of [¹⁴C]-urea into dur3Δ yeast cells expressing one of several urea transporters identified during screening were performed essentially as previously described with slight modifications (Morel, et al., Fungal Genetics and Biology 2008). dur3A yeast cells were grown to mid log phase before harvesting and resuspending in uptake buffer. Cells were then incubated with the 0 μM to 200 μM of [¹⁴C] spiked urea and allowed to incubate in the presences of substrate for four minutes. Cells were then washed and a liquid scintillation counter was used to determine the amount of urea taken into the cells. As seen in FIG. 5, many of the transporters dramatically increased the amount of labeled urea taken into the cell even at extracellular concentrations as low as 10 μM and during relatively short times.

FIG. 10: Constructs designed for co-expression of two urea transporters from both a constitutive and a root preferred promoter have been designed and created. These constructs are to be transformed into both Arabidopsis and maize Gaspe germplasm using a previously established Agrobacterium mediated transformation protocol. Positive transgene events will be assessed for general growth and reproductive parameters as well as screened for an increased ability to use urea as a nitrogen source. Transgenic plants will be assessed for the potential of enhanced urea uptake at both low and high levels of extracellular urea and significant events would be expected to increase uptake and N status of the cell over a wide range of urea in the growth medium as illustrated in the figure.

FIG. 11: To illustrate the impact of manipulation of these genes in transgenic corn, field tests have been conducted. Progeny seed of multiple transgenic events for a single transformation vector, PHP45645, were planted in the field to evaluate the transgenes' ability to enhance yield/NUE under normal (NN) and reduced soil (LN) nitrogen as compared to the non-transgenic control plants (BN). This vector contains sequence encoding the native maize DUR3 polypeptide (SEQ ID NO: 3) under the control of the root preferred RM2 promoter as well as the maize GS1-3 (SEQ ID NO: 301) and the urease (SEQ ID NO: 322) polypeptides under the control of the PEPC promoter. These experiments were conducted at multiple locations with multiple replicates, and the data collected measured plant yield. The experimental data, presented as average bushels per acre yield, reveal the transgenic corn plants perform equivalent to non-transgenic control plants under low N conditions, however yield is slightly reduced for transgenic plants compared to non-transgenic controls under normal N field conditions. These results indicate that overexpression of the maize Dur3 protein, earner characterized as a relatively poor urea transporter using yeast uptake assays, might not be sufficient to enhance urea uptake in a field environment. Additional yield trials will also be conducted using similar stack constructs containing the more efficient urea transporters described in this text.

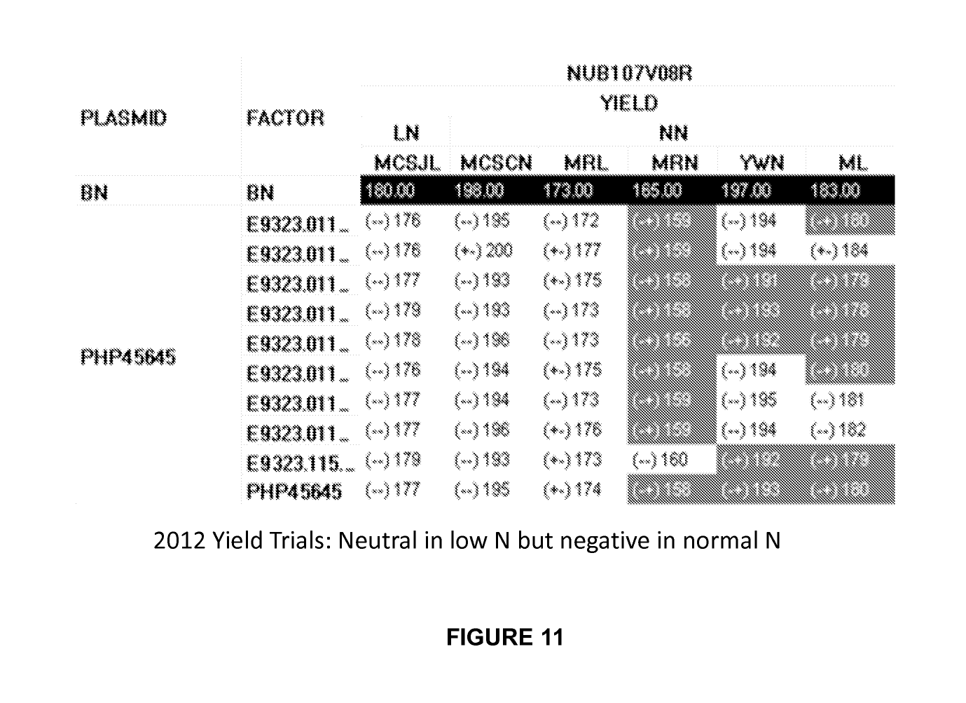

### DETAILED DESCRIPTION

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature. See, e.g., Langenheim and Thimann, BOTANY: PLANT BIOLOGY AND ITS RELATION TO HUMAN AFFAIRS, John Wiley (1982); CELL CULTURE AND SOMATIC CELL GENETICS OF PLANTS, vol. 1, Vasil, ed. (1984); Stanier, et al., THE MICROBIAL WORLD, 5^{th} ed., Prentice-Hall (1986); Dhringra and Sinclair, BASIC PLANT PATHOLOGY METHODS, CRC Press (1985); Maniatis, et al., MOLECULAR CLONING: A LABORATORY MANUAL (1982); DNA CLONING, vols. I and II, Glover, ed. (1985); OLIGONUCLEOTIDE SYNTHESIS, Gait, ed. (1984); NUCLEIC ACID HYBRIDIZATION, Hames and Higgins, eds. (1984) and the series METHODS IN ENZYMOLOGY, Colowick and Kaplan, eds, Academic Press, Inc., San Diego, Calif.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present invention, the following terms will be employed and are intended to be defined as indicated below.

By “microbe” is meant any microorganism (including both eukaryotic and prokaryotic microorganisms), such as fungi, yeast, bacteria, actinomycetes, algae and protozoa, as well as other unicellular structures.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., DIAGNOSTIC MOLECULAR MICROBIOLOGY: PRINCIPLES AND APPLICATIONS, Persing, et al., eds., American Society for Microbiology, Washington, D.C. (1993). The product of amplification is termed an amplicon.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32) can be modified to yield a functionally identical molecule. Accordingly, each silent variation of a nucleic acid, which encodes a polypeptide of the present invention, is implicit in each described polypeptide sequence and incorporated herein by reference.

As to amino acid sequences, one of skill will recognize that individual substitutions, deletions or additions to a nucleic acid, peptide, polypeptide or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity or ligand/receptor binding is generally at least 30%, 40%, 50%, 60%, 70%, 80% or 90%, preferably 60-90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V) and

6) Phenylalanine (F), Tyrosine (Y), Tryptophan (W).

See also, Creighton, PROTEINS, W.H. Freeman and Co. (1984).

As used herein, “consisting essentially of” means the inclusion of additional sequences to an object polynucleotide where the additional sequences do not selectively hybridize, under stringent hybridization conditions, to the same cDNA as the polynucleotide and where the hybridization conditions include a wash step in 0.1×SSC and 0.1% sodium dodecyl sulfate at 65° C.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolumn (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98, and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which comprises a heterologous nucleic acid sequence of the invention, which contains a vector and supports the replication and/or expression of the expression vector. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet, switchgrass, myscanthus, triticale and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected mRNA).

The terms “isolated” refers to material, such as a nucleic acid or a protein, which is substantially or essentially free from components which normally accompany or interact with it as found in its naturally occurring environment. The isolated material optionally comprises material not found with the material in its natural environment. Nucleic acids, which are “isolated”, as defined herein, are also referred to as “heterologous” nucleic acids. Unless otherwise stated, the term “GS nucleic acid” means a nucleic acid comprising a polynucleotide (“GS polynucleotide”) encoding a full length or partial length Glutamine Synthetase polypeptide.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, GUIDE TO MOLECULAR CLONING TECHNIQUES, from the series METHODS IN ENZYMOLOGY, vol. 152, Academic Press, Inc., San Diego, Calif. (1987); Sambrook, et al., MOLECULAR CLONING: A LABORATORY MANUAL, 2^{nd} ed., vols. 1-3 (1989) and CURRENT PROTOCOLS IN MOLECULAR BIOLOGY, Ausubel, et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter and a second sequence, wherein the promoter sequence initiates and mediates transcription of the DNA corresponding to the second sequence.

Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen and microspores. The class of plants, which can be used in the methods of the invention, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is Zea mays.

As used herein, “yield” may include reference to bushels per acre of a grain crop at harvest, as adjusted for grain moisture (15% typically for maize, for example). Grain moisture is measured in the grain at harvest. The adjusted test weight of grain is determined to be the weight in pounds per bushel, adjusted for grain moisture level at harvest.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Examples are promoters that preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions.

The term “GS polypeptide” refers to one or more amino acid sequences. The term is also inclusive of fragments, variants, homologs, alleles or precursors (e.g., preproproteins or proproteins) thereof. A “GS protein” comprises a Glutamine Synthetase polypeptide. Unless otherwise stated, the term “GS nucleic acid” means a nucleic acid comprising a polynucleotide (“GS polynucleotide”) encoding a Glutamine Synthetase polypeptide.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention; or may have reduced or eliminated expression of a native gene. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed, and a promoter.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass known analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1M NaCl, 1% SDS at 37° C. and a wash in 0.5× to 1×SSC at 55 to 60° C. Exemplary high stringency conditions include hybridization in 50% formamide, 1M NaCl, 1% SDS at 37° C. and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem., 138:267-84: Tₘ=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, LABORATORY TECHNIQUES IN BIOCHEMISTRY AND MOLECULAR BIOLOGY—HYBRIDIZATION WITH NUCLEIC ACID PROBES, part I, chapter 2, “Overview of principles of hybridization and the strategy of nucleic acid probe assays,” Elsevier, New York (1993); and CURRENT PROTOCOLS IN MOLECULAR BIOLOGY, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA and 25 mM Na phosphate at 65° C., and a wash in 0.1×SSC, 0.1% SDS at 65° C.

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package®, Version 8 (available from Genetics Computer Group (GCG® programs (Accelrys, Inc., San Diego, Calif.)). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65, and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, CURRENT PROTOCOLS IN MOLECULAR BIOLOGY, Chapter 19, Ausubel, et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package® are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package® is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

The invention discloses urea transporter, urease and glutamine synthetase polynucleotides and polypeptides. The novel nucleotides and proteins of the invention have an expression pattern which indicates that they regulate nitrogen transport and thus play an important role in plant development. The polynucleotides are expressed in various plant tissues. The polynucleotides and polypeptides thus provide an opportunity to manipulate plant development to alter seed and vegetative tissue development, timing or composition. This may be used to create a plant with altered N composition in source and sink.

### Nucleic Acids

The present invention provides, inter alia, isolated nucleic acids of RNA, DNA and analogs and/or chimeras thereof, comprising a urea transporter, urease or glutamine synthetase polynucleotide.

The present invention also includes polynucleotides optimized for expression in different organisms. For example, for expression of the polynucleotide in a maize plant, the sequence can be altered to account for specific codon preferences and to alter GC content as according to Murray, et al., supra. Maize codon usage for 28 genes from maize plants is listed in Table 4 of Murray et al., supra.

The urea transporter, urease, and glutamine synthetase nucleic acids of the present invention comprise isolated urea transporter, urease and glutamine synthetase polynucleotides which are inclusive of:
(a) a polynucleotide encoding a urea transporter, urease or glutamine synthetase polypeptide and conservatively modified and polymorphic variants thereof;
(b) a polynucleotide having at least 70% sequence identity with polynucleotides of (a) or (b);
(c) complementary sequences of polynucleotides of (a) or (b).

### Construction of Nucleic Acids

The isolated nucleic acids of the present invention can be made using (a) standard recombinant methods, (b) synthetic techniques or combinations thereof. In some embodiments, the polynucleotides of the present invention will be cloned, amplified or otherwise constructed from a fungus or bacteria.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. The nucleic acid of the present invention—excluding the polynucleotide sequence—is optionally a vector, adapter or linker for cloning and/or expression of a polynucleotide of the present invention. Additional sequences may be added to such cloning and/or expression sequences to optimize their function in cloning and/or expression, to aid in isolation of the polynucleotide or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters and linkers is well known in the art. Exemplary nucleic acids include such vectors as: M13, lambda ZAP Express, lambda ZAP II, lambda gt10, lambda gt11, pBK-CMV, pBK-RSV, pBluescript II, lambda DASH II, lambda EMBL 3, lambda EMBL 4, pWE15, SuperCos 1, SurfZap, Uni-ZAP, pBC, pBS+/−, pSG5, pBK, pCR-Script, pET, pSPUTK, p3′SS, pGEM, pSK+/−, pGEX, pSPORTI and II, pOPRSVI CAT, pOPl3 CAT, pXT1, pSG5, pPbac, pMbac, pMC1neo, pOG44, pOG45, pFRTβGAL, pNEOβGAL, pRS403, pRS404, pRS405, pRS406, pRS413, pRS414, pRS415, pRS416, lambda MOSSIox and lambda MOSElox. Optional vectors for the present invention, include but are not limited to, lambda ZAP II and pGEX. For a description of various nucleic acids see, e.g., Stratagene Cloning Systems, Catalogs 1995, 1996, 1997 (La Jolla, Calif.); and, Amersham Life Sciences, Inc, Catalog '97 (Arlington Heights, Ill.).

### Synthetic Methods for Constructing Nucleic Acids

The isolated nucleic acids of the present invention can also be prepared by direct chemical synthesis by methods such as the phosphotriester method of Narang, et al., (1979) Meth. Enzymol. 68:90-9; the phosphodiester method of Brown, et al., (1979) Meth. Enzymol. 68:109-51; the diethylphosphoramidite method of Beaucage, et al., (1981) Tetra. Letts. 22(20):1859-62; the solid phase phosphoramidite triester method described by Beaucage, et al., supra, e.g., using an automated synthesizer, e.g., as described in Needham-VanDevanter, et al., (1984) Nucleic Acids Res. 12:6159-68 and the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G>7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present invention provides 5′ and/or 3′ UTR regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present invention can be analyzed statistically using commercially available software packages such as “Codon Preference” available from the University of Wisconsin Genetics Computer Group. See, Devereaux, et al., (1984) Nucleic Acids Res. 12:387-395 or MacVector 4.1 (Eastman Kodak Co., New Haven, Conn.). Thus, the present invention provides a codon usage frequency characteristic of the coding region of at least one of the polynucleotides of the present invention. The number of polynucleotides (3 nucleotides per amino acid) that can be used to determine a codon usage frequency can be any integer from 3 to the number of polynucleotides of the present invention as provided herein. Optionally, the polynucleotides will be full-length sequences. An exemplary number of sequences for statistical analysis can be at least 1, 5, 10, 20, 50 or 100.

### Sequence Shuffling

The present invention provides methods for sequence shuffling using polynucleotides of the present invention, and compositions resulting therefrom. Sequence shuffling is described in PCT Publication Number 1996/19256. See also, Zhang, et al., (1997) Proc. Natl. Acad. Sci. USA 94:4504-9 and Zhao, et al., (1998) Nature Biotech 16:258-61. Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation or other expression property of a gene or transgene, a replicative element, a protein-binding element, or the like, such as any feature which confers a selectable or detectable property. In some embodiments, the selected characteristic will be an altered Kₘ and/or K_{cat} over the wild-type protein as provided herein. In other embodiments, a protein or polynucleotide generated from sequence shuffling will have a ligand binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

### Recombinant Expression Cassettes

The present invention further provides recombinant expression cassettes comprising a nucleic acid of the present invention. A nucleic acid sequence coding for the desired polynucleotide of the present invention, for example a cDNA or a genomic sequence encoding a polypeptide long enough to code for an active protein of the present invention, can be used to construct a recombinant expression cassette which can be introduced into the desired host cell. A recombinant expression cassette will typically comprise a polynucleotide of the present invention operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated or cell- or tissue-specific/selective expression), a transcription initiation start site, a ribosome binding site, an RNA processing signal, a transcription termination site and/or a polyadenylation signal.

A plant promoter fragment can be employed which will direct expression of a polynucleotide of the present invention in all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30) and maize H3 histone (Lepetit, et al., (1992) Mol. Gen. Genet. 231:276-85 and Atanassvoa, et al., (1992) Plant Journal 2(3):291-300); ALS promoter, as described in PCT Application Number WO 1996/30530 and other transcription initiation regions from various plant genes known to those of skill. For the present invention ubiquitin is the preferred promoter for expression in monocot plants.

Alternatively, the plant promoter can direct expression of a polynucleotide of the present invention in a specific tissue or may be otherwise under more precise environmental or developmental control. Such promoters are referred to here as “inducible” promoters. Environmental conditions that may affect transcription by inducible promoters include pathogen attack, anaerobic conditions or the presence of light. Examples of inducible promoters are the Adh1 promoter, which is inducible by hypoxia or cold stress, the Hsp70 promoter, which is inducible by heat stress and the PPDK promoter, which is inducible by light.

Examples of promoters under developmental control include promoters that initiate transcription only, or preferentially, in certain tissues, such as leaves, roots, fruit, seeds or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of plant genes or from T-DNA. The 3′ end sequence to be added can be derived from, for example, the nopaline synthase or octopine synthase genes or alternatively from another plant gene or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50 and An, et al., (1989) Plant Cell 1:115-22) and the CaMV 19S gene (Mogen, et al., (1990) Plant Cell 2:1261-72).

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2, and 6, the Bronze-1 intron are known in the art. See generally, THE MAIZE HANDBOOK, Chapter 116, Freeling and Walbot, eds., Springer, New York (1994).

Plant signal sequences, including, but not limited to, signal-peptide encoding DNA/RNA sequences which target proteins to the extracellular matrix of the plant cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant Cell, 2:301-13); signal peptides which cause proteins to be secreted, such as that of PR1b (Lind, et al., (1992) Plant Mol. Biol. 18:47-53) or the barley alpha amylase (BAA) (Rahmatullah, et al., (1989) Plant Mol. Biol. 12:119, and hereby incorporated by reference) or signal peptides which target proteins to the plastids such as that of rapeseed enoyl-Acp reductase (Verwaert, et al., (1994) Plant Mol. Biol. 26:189-202) are useful in the invention. The barley alpha amylase signal sequence fused to the urease polynucleotide is the preferred construct for expression in maize for the present invention.

The vector comprising the sequences from a polynucleotide of the present invention will typically comprise a marker gene, which confers a selectable phenotype on plant cells. Usually, the selectable marker gene will encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene), or other such genes known in the art. The bar gene encodes resistance to the herbicide basta and the ALS gene encodes resistance to the herbicide chlorsulfuron.

Typical vectors useful for expression of genes in higher plants are well known in the art and include vectors derived from the tumor-inducing (Ti) plasmid of Agrobacterium tumefaciens described by Rogers, et al., (1987) Meth. Enzymol. 153:253-77. These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl, et al., (1987) Gene 61:1-11 and Berger, et al., (1989) Proc. Natl. Acad. Sci. USA, 86:8402-6. Another useful vector herein is plasmid pBI101.2 that is available from CLONTECH Laboratories, Inc. (Palo Alto, Calif.).

### Expression of Proteins in Host Cells

Using the nucleic acids of the present invention, one may express a protein of the present invention in a recombinantly engineered cell such as bacteria, yeast, insect, mammalian or preferably plant cells. The cells produce the protein in a non-natural condition (e.g., in quantity, composition, location and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding a protein of the present invention will typically be achieved by operably linking, for example, the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences and promoters useful for regulation of the expression of the DNA encoding a protein of the present invention. To obtain high level expression of a cloned gene, it is desirable to construct expression vectors which contain, at the minimum, a strong promoter, such as ubiquitin, to direct transcription, a ribosome binding site for translational initiation and a transcription/translation terminator. Constitutive promoters are classified as providing for a range of constitutive expression. Thus, some are weak constitutive promoters and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level,” or about 1/10 transcripts to about 1/100 transcripts to about 1/1,000 transcripts.

One of skill would recognize that modifications could be made to a protein of the present invention without diminishing its biological activity. Some modifications may be made to facilitate the cloning, expression or incorporation of the targeting molecule into a fusion protein. Such modifications are well known to those of skill in the art and include, for example, a methionine added at the amino terminus to provide an initiation site or additional amino acids (e.g., poly His) placed on either terminus to create conveniently located restriction sites or termination codons or purification sequences.

### Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present invention.

### Expression in Eukaryotes

A variety of eukaryotic expression systems such as yeast, insect cell lines, plant and mammalian cells, are known to those of skill in the art. As explained briefly below, the present invention can be expressed in these eukaryotic systems. In some embodiments, transformed/transfected plant cells, as discussed infra, are employed as expression systems for production of the proteins of the instant invention.

Synthesis of heterologous proteins in yeast is well known. Sherman, et al., METHODS IN YEAST GENETICS, Cold Spring Harbor Laboratory (1982) is a well recognized work describing the various methods available to produce the protein in yeast. Two widely utilized yeasts for production of eukaryotic proteins are Saccharomyces cerevisiae and Pichia pastoris. Vectors, strains and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase and an origin of replication, termination sequences and the like as desired.

A protein of the present invention, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins of the present invention can also be ligated to various expression vectors for use in transfecting cell cultures of, for instance, mammalian, insect or plant origin. Mammalian cell systems often will be in the form of monolayers of cells although mammalian cell suspensions may also be used. A number of suitable host cell lines capable of expressing intact proteins have been developed in the art, and include the HEK293, BHK21 and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:49) and necessary processing information sites, such as ribosome binding sites, RNA splice sites, polyadenylation sites (e.g., an SV40 large T Ag poly A addition site) and transcriptional terminator sequences. Other animal cells useful for production of proteins of the present invention are available, for instance, from the American Type Culture Collection Catalogue of Cell Lines and Hybridomas (7^{th} ed., 1992).

Appropriate vectors for expressing proteins of the present invention in insect cells are usually derived from the SF9 baculovirus. Suitable insect cell lines include mosquito larvae, silkworm, armyworm, moth and Drosophila cell lines such as a Schneider cell line (see, e.g., Schneider, (1987) J. Embryol. Exp. Morphol. 27:353-65).

As with yeast, when higher animal or plant host cells are employed, polyadenlyation or transcription terminator sequences are typically incorporated into the vector. An example of a terminator sequence is the polyadenlyation sequence from the bovine growth hormone gene. Sequences for accurate splicing of the transcript may also be included. An example of a splicing sequence is the VP1 intron from SV40 (Sprague, et al., (1983) J. Virol. 45:773-81). Additionally, gene sequences to control replication in the host cell may be incorporated into the vector such as those found in bovine papilloma virus type-vectors (Saveria-Campo, “Bovine Papilloma Virus DNA a Eukaryotic Cloning Vector,” in DNA CLONING: A PRACTICAL APPROACH, vol. II, Glover, ed., IRL Press, Arlington, Va., pp. 213-38 (1985)).

In addition, the gene for a urea transporter, urease, or glutamine synthetase placed in the appropriate plant expression vector can be used to transform plant cells. The polypeptide can then be isolated from plant callus or the transformed cells can be used to regenerate transgenic plants. Such transgenic plants can be harvested, and the appropriate tissues (seed or leaves, for example) can be subjected to large scale protein extraction and purification techniques.

### Plant Transformation Methods

Numerous methods for introducing foreign genes into plants are known and can be used to insert a urea transporter, urease, or glutamine synthetase polynucleotide into a plant host, including biological and physical plant transformation protocols. See, e.g., Miki et al., “Procedure for Introducing Foreign DNA into Plants,” in METHODS IN PLANT MOLECULAR BIOLOGY AND BIOTECHNOLOGY, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch, et al., (1985) Science 227:1229-31), electroporation, micro-injection and biolistic bombardment.

Expression cassettes and vectors and in vitro culture methods for plant cell or tissue transformation and regeneration of plants are known and available. See, e.g., Gruber, et al., “Vectors for Plant Transformation,” in METHODS IN PLANT MOLECULAR BIOLOGY AND BIOTECHNOLOGY, supra, pp. 89-119.

The isolated polynucleotides or polypeptides may be introduced into the plant by one or more techniques typically used for direct delivery into cells. Such protocols may vary depending on the type of organism, cell, plant or plant cell, i.e. monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski, et al., (1984) EMBO J. 3:2717-2722) and ballistic particle acceleration (see, for example, Sanford, et al., U.S. Pat. No. 4,945,050; WO 1991/10725 and McCabe, et al., (1988) Biotechnology 6:923-926). Also see, Tomes, et al., Direct DNA Transfer into Intact Plant Cells Via Microprojectile Bombardment. pp. 197-213 in Plant Cell, Tissue and Organ Culture, Fundamental Methods. eds. Gamborg and Phillips, Springer-Verlag Berlin Heidelberg New York, 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 1991/10725 (maize); Klein, et al., (1988) Plant Physiol. 91:440-444 (maize); Fromm, et al., (1990) Biotechnology 8:833-839 and Gordon-Kamm, et al., (1990) Plant Ce112:603-618 (maize); Hooydaas-Van Slogteren and Hooykaas, (1984) Nature (London) 311:763-764; Bytebier, et al., (1987) Proc. Natl. Acad. Sci. USA 84:5345-5349 (Liliaceae); De Wet, et al., (1985) In The Experimental Manipulation of Ovule Tissues, ed. Chapman, et al., pp. 197-209 Longman, N Y (pollen); Kaeppler, et al., (1990) Plant Cell Reports 9:415-418 and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255 and Christou and Ford (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185), all of which are herein incorporated by reference.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. Pat. No. 913,914, filed Oct. 1, 1986, as referenced in U.S. Pat. No. 5,262,306, issued Nov. 16, 1993 and Simpson, et al., (1986) Plant Mol. Biol. 6:403-15 (also referenced in the '306 patent), all incorporated by reference in their entirety.

Once constructed, these plasmids can be placed into A. rhizogenes or A. tumefaciens and these vectors used to transform cells of plant species, which are ordinarily susceptible to Fusarium or Alternaria infection. Several other transgenic plants are also contemplated by the present invention including but not limited to soybean, corn, sorghum, alfalfa, rice, clover, cabbage, banana, coffee, celery, tobacco, cowpea, cotton, melon, switchgrass, myscanthus, triticale and pepper. The selection of either A. tumefaciens or A. rhizogenes will depend on the plant being transformed thereby. In general A. tumefaciens is the preferred organism for transformation. Most dicotyledonous plants, some gymnosperms, and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae and Chenopodiaceae. Monocot plants can now be transformed with some success. EP Patent Application Number 604 662 A1 discloses a method for transforming monocots using Agrobacterium. EP Patent Application Number 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, (1985) Theor. Appl. Genet. 69:235-40; U.S. Pat. No. 4,658,082; Simpson, et al., supra and U.S. Pat. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206 and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731 and Christou, et al., (1987) Proc. Natl. Acad. Sci. USA 84:3962. Direct uptake of DNA into protoplasts using CaCl₂ precipitation, polyvinyl alcohol or poly-L-ornithine has also been reported. See, e.g., Hain, et al., (1985) Mol. Gen. Genet. 199:161 and Draper, et al., (1982) Plant Cell Physiol. 23:451.

Electroporation of protoplasts and whole cells and tissues has also been described. See, e.g., Donn, et al., (1990) in Abstracts of the VIIth Int'l. Congress on Plant Cell and Tissue Culture IAPTC, A2-38, p. 53; D'Halluin, et al., (1992) Plant Cell 4:1495-505 and Spencer, et al., (1994) Plant Mol. Biol. 24:51-61.

Increasing the Activity and/or Level of a Urea Transporter, Urease or Glutamine Synthetase Polypeptide

Methods are provided to increase the activity and/or level of the urea transporter, urease, and glutamine synthetase polypeptides of the invention. An increase in the level and/or activity of the urea transporter, urease, or glutamine synthetase polypeptide of the invention can be achieved by providing to the plant a urea transporter, urease or glutamine synthetase polypeptide. The urea transporter, urease or glutamine synthetase polypeptide can be provided by introducing the amino acid sequence encoding the urea transporter, urease or glutamine synthetase polypeptide into the plant, introducing into the plant a nucleotide sequence encoding a urea transporter, urease or glutamine synthetase polypeptide or alternatively by modifying a genomic locus encoding the urea transporter, urease or glutamine synthetase polypeptide of the invention.

As discussed elsewhere herein, many methods are known the art for providing a polypeptide to a plant including, but not limited to, direct introduction of the polypeptide into the plant, introducing into the plant (transiently or stably) a polynucleotide construct encoding a polypeptide having urea transport, urea breakdown or ammonia assimilation activity. It is also recognized that the methods of the invention may employ a polynucleotide that is not capable of directing, in the transformed plant, the expression of a protein or an RNA. Thus, the level and/or activity of a urea transporter, urease, or glutamine synthetase polypeptide may be increased by altering the gene encoding the urea transporter, urease, or glutamine synthetase polypeptide or its promoter. See, e.g., Kmiec, U.S. Pat. No. 5,565,350; Zarling, et al., PCT/US93/03868. Therefore mutagenized plants that carry mutations in urea transporter, urease, or glutamine synthetase genes, where the mutations increase expression of the urea transporter, urease, or glutamine synthetase gene or increase the urea transport, urea breakdown, or ammonia assimilation activity of the encoded polypeptide are provided.

### Modulating Root Development

Methods for modulating root development in a plant are provided. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development or radial expansion.

Methods for modulating root development in a plant are provided. The methods comprise modulating the level and/or activity of the urea transporter, urease and glutamine synthetase polypeptide in the plant. In one method, a urea transporter, urease or glutamine synthetase sequence of the invention is provided to the plant. In another method, the urea transporter, urease, or glutamine synthetase nucleotide sequence is provided by introducing into the plant a polynucleotide comprising a urea transporter, urease or glutamine synthetase nucleotide sequence of the invention, expressing the urea transporter, urease or glutamine synthetase sequence and thereby modifying root development. In still other methods, the urea transporter, urease or glutamine synthetase nucleotide construct introduced into the plant is stably incorporated into the genome of the plant.

In other methods, root development is modulated by altering the level or activity of the urea transporter, urease or glutamine synthetase polypeptide in the plant. A change in urea transport, urea breakdown or ammonia assimilation activity can result in at least one or more of the following alterations to root development, including, but not limited to, larger root meristems, increased in root growth, enhanced radial expansion, an enhanced vasculature system, increased root branching, more adventitious roots and/or an increase in fresh root weight when compared to a control plant.

As used herein, “root growth” encompasses all aspects of growth of the different parts that make up the root system at different stages of its development in both monocotyledonous and dicotyledonous plants. It is to be understood that enhanced root growth can result from enhanced growth of one or more of its parts including the primary root, lateral roots, adventitious roots, etc.

Methods of measuring such developmental alterations in the root system are known in the art. See, for example, US Patent Application Publication Number 2003/0074698 and Werner, et al., (2001) PNAS 18:10487-10492, both of which are herein incorporated by reference.

As discussed above, one of skill will recognize the appropriate promoter to use to modulate root development in the plant. Exemplary promoters for this embodiment include constitutive promoters and root-preferred promoters. Exemplary root-preferred promoters have been disclosed elsewhere herein.

Stimulating root growth and increasing root mass by modulating the activity and/or level of the urea transporter, urease, or glutamine synthetase polypeptide also finds use in improving the standability of a plant. The term “resistance to lodging” or “standability” refers to the ability of a plant to fix itself to the soil. For plants with an erect or semi-erect growth habit, this term also refers to the ability to maintain an upright position under adverse (environmental) conditions. This trait relates to the size, depth and morphology of the root system. In addition, stimulating root growth and increasing root mass by modulating the level and/or activity of the urea transporter, urease, or glutamine synthetase polypeptide also finds use in promoting in vitro propagation of explants.

Furthermore, higher root biomass production due to an increased level and/or activity of urea transport, urea breakdown or ammonia assimilation activity has a direct effect on the yield and an indirect effect of production of compounds produced by root cells or transgenic root cells or cell cultures of said transgenic root cells. One example of an interesting compound produced in root cultures is shikonin, the yield of which can be advantageously enhanced by said methods.

Accordingly, the present invention further provides plants having modulated root development when compared to the root development of a control plant. In some embodiments, the plant of the invention has an increased level/activity of the urea transporter, urease or glutamine synthetase polypeptide of the invention and has enhanced root growth and/or root biomass. In other embodiments, such plants have stably incorporated into their genome a nucleic acid molecule comprising a urea transporter, urease or glutamine synthetase nucleotide sequence of the invention operably linked to a promoter that drives expression in the plant cell.

### Modulating Shoot and Leaf Development

Methods are also provided for modulating shoot and leaf development in a plant. By “modulating shoot and/or leaf development” is intended any alteration in the development of the plant shoot and/or leaf. Such alterations in shoot and/or leaf development include, but are not limited to, alterations in shoot meristem development, in leaf number, leaf size, leaf and stem vasculature, internode length and leaf senescence. As used herein, “leaf development” and “shoot development” encompasses all aspects of growth of the different parts that make up the leaf system and the shoot system, respectively, at different stages of their development, both in monocotyledonous and dicotyledonous plants. Methods for measuring such developmental alterations in the shoot and leaf system are known in the art. See, for example, Werner, et al., (2001) PNAS 98:10487-10492 and US Patent Application Publication Number 2003/0074698, each of which is herein incorporated by reference.

The method for modulating shoot and/or leaf development in a plant comprises modulating the activity and/or level of a urea transporter, urease or glutamine synthetase polypeptide of the invention. In one embodiment, a urea transporter, urease or glutamine synthetase sequence of the invention is provided. In other embodiments, the urea transporter, urease or glutamine synthetase nucleotide sequence can be provided by introducing into the plant a polynucleotide comprising a urea transporter, urease or glutamine synthetase nucleotide sequence of the invention, expressing the urea transporter, urease or glutamine synthetase sequence and thereby modifying shoot and/or leaf development. In other embodiments, the urea transporter, urease or glutamine synthetase nucleotide construct introduced into the plant is stably incorporated into the genome of the plant.

In specific embodiments, shoot or leaf development is modulated by increasing the level and/or activity of the urea transporter, urease or glutamine synthetase polypeptide in the plant. An increase in urea transport, urea breakdown or ammonia assimilation activity can result in at least one or more of the following alterations in shoot and/or leaf development, including, but not limited to, leaf number, leaf surface, vasculature, internode length and leaf senescence, when compared to a control plant.

As discussed above, one of skill will recognize the appropriate promoter to use to modulate shoot and leaf development of the plant. Exemplary promoters for this embodiment include constitutive promoters, shoot-preferred promoters, shoot meristem-preferred promoters and leaf-preferred promoters. Exemplary promoters have been disclosed elsewhere herein.

As discussed above, modulation urea transport, urea breakdown or ammonia assimilation activity in the plant modulates both root and shoot growth. Thus, the present invention further provides methods for altering the root/shoot ratio.

Accordingly, the present invention further provides plants having modulated shoot and/or leaf development when compared to a control plant. In some embodiments, the plant of the invention has an increased level/activity of the urea transporter, urease or glutamine synthetase polypeptide of the invention. In other embodiments, the plant of the invention has a decreased level/activity of the urea transporter, urease or glutamine synthetase polypeptide of the invention.

### Modulating Reproductive Tissue Development

Methods for modulating reproductive tissue development are provided. In one embodiment, methods are provided to modulate floral development in a plant. By “modulating floral development” is intended any alteration in a structure of a plant's reproductive tissue as compared to a control plant in which the activity or level of the urea transporter, urease, or glutamine synthetase polypeptide has not been modulated. “Modulating floral development” further includes any alteration in the timing of the development of a plant's reproductive tissue (i.e., a delayed or a accelerated timing of floral development) when compared to a control plant in which the activity or level of the urea transporter, urease or glutamine synthetase polypeptide has not been modulated. Macroscopic alterations may include changes in size, shape, number or location of reproductive organs, the developmental time period that these structures form or the ability to maintain or proceed through the flowering process in times of environmental stress. Microscopic alterations may include changes to the types or shapes of cells that make up the reproductive organs.

The method for modulating floral development in a plant comprises modulating urea transport, urea breakdown, or ammonia assimilation activity in a plant. In one method, a urea transporter, urease or glutamine synthetase sequence of the invention is provided. A urea transporter, urease or glutamine synthetase nucleotide sequence can be provided by introducing into the plant a polynucleotide comprising a urea transporter, urease or glutamine synthetase nucleotide sequence of the invention, expressing the urea transporter, urease or glutamine synthetase sequence and thereby modifying floral development. In other embodiments, the urea transporter, urease or glutamine synthetase nucleotide construct introduced into the plant is stably incorporated into the genome of the plant.

In specific methods, floral development is modulated by increasing the level or activity of the urea transporter, urease or glutamine synthetase polypeptide in the plant. An increase in urea transport, urea breakdown or ammonia assimilation activity can result in at least one or more of the following alterations in floral development, including, but not limited to, retarded flowering, reduced number of flowers, partial male sterility and reduced seed set, when compared to a control plant. Inducing delayed flowering or inhibiting flowering can be used to enhance yield in forage crops such as alfalfa. Methods for measuring such developmental alterations in floral development are known in the art. See, for example, Mouradov, et al., (2002) The Plant Cell S111-S130, herein incorporated by reference.

As discussed above, one of skill will recognize the appropriate promoter to use to modulate floral development of the plant. Exemplary promoters for this embodiment include constitutive promoters, inducible promoters, shoot-preferred promoters and inflorescence-preferred promoters.

Methods are also provided for the use of the urea transporter, urease or glutamine synthetase sequences of the invention to increase nitrogen use efficiency. The method comprises decreasing or increasing the activity of the urea transporter, urease or glutamine synthetase sequences in a plant or plant part, such as the roots, shoot, epidermal cells, etc.

As discussed above, one of skill will recognize the appropriate promoter to use to manipulate the expression of GS. Exemplary promoters of this embodiment include constitutive promoters, inducible promoters and root or shoot or leaf preferred promoters.

### Method of Use for Urea Transporter, Urease or Glutamine Synthetase Promoter Polynucleotides

The polynucleotides comprising the urea transporter, urease or glutamine synthetase promoters disclosed in the present invention, as well as variants and fragments thereof, are useful in the genetic manipulation of any host cell, preferably plant cell, when assembled with a DNA construct such that the promoter sequence is operably linked to a nucleotide sequence comprising a polynucleotide of interest. In this manner, the urea transporter, urease or glutamine synthetase promoter polynucleotides of the invention are provided in expression cassettes along with a polynucleotide sequence of interest for expression in the host cell of interest. Urea transporter, urease or glutamine synthetase promoter sequences of the invention are expressed in a variety of tissues and thus the promoter sequences can find use in regulating the temporal and/or the spatial expression of polynucleotides of interest.

Synthetic hybrid promoter regions are known in the art. Such regions comprise upstream promoter elements of one polynucleotide operably linked to the promoter element of another polynucleotide. In an embodiment of the invention, heterologous sequence expression is controlled by a synthetic hybrid promoter comprising the urea transporter, urease or glutamine synthetase promoter sequences of the invention or a variant or fragment thereof, operably linked to upstream promoter element(s) from a heterologous promoter. Upstream promoter elements that are involved in the plant defense system have been identified and may be used to generate a synthetic promoter. See, for example, Rushton, et al., (1998) Curr. Opin. Plant Biol. 1:311-315. Alternatively, a synthetic urea transporter, urease or glutamine synthetase promoter sequence may comprise duplications of the upstream promoter elements found within the urea transporter, urease or glutamine synthetase promoter sequences.

It is recognized that the promoter sequence of the invention may be used with its native urea transporter, urease or glutamine synthetase coding sequences. A DNA construct comprising the urea transporter, urease or glutamine synthetase promoter operably linked with its native urea transporter, urease or glutamine synthetase gene may be used to transform any plant of interest to bring about a desired phenotypic change, such as, modulating root, shoot, leaf, floral and embryo development, stress tolerance and any other phenotype described elsewhere herein.

The promoter nucleotide sequences and methods disclosed herein are useful in regulating expression of any heterologous nucleotide sequence in a host plant in order to vary the phenotype of a plant. Various changes in phenotype are of interest including modifying the fatty acid composition in a plant, altering the amino acid content of a plant, altering a plant's pathogen defense mechanism and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as understanding of agronomic traits and characteristics such as yield and heterosis increase, the choice of genes for transformation will change accordingly. General categories of genes of interest include, for example, those genes involved in information, such as GSs, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics and commercial products. Genes of interest include, generally, those involved in oil, starch, carbohydrate or nutrient metabolism as well as those affecting kernel size, sucrose loading and the like.

In certain embodiments the nucleic acid sequences of the present invention can be used in combination (“stacked”) with other polynucleotide sequences of interest in order to create plants with a desired phenotype. The combinations generated can include multiple copies of any one or more of the polynucleotides of interest. The polynucleotides of the present invention may be stacked with any gene or combination of genes to produce plants with a variety of desired trait combinations, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802 and 5,703,409); barley high lysine (Williamson, et al., (1987) Eur. J. Biochem. 165:99-106 and WO 1998/20122) and high methionine proteins (Pedersen, et al., (1986) J. Biol. Chem. 261:6279; Kirihara, et al., (1988) Gene 71:359 and Musumura, et al., (1989) Plant Mol. Biol. 12:123)); increased digestibility (e.g., modified storage proteins (U.S. patent application Ser. No. 10/053,410, filed Nov. 7, 2001) and thioredoxins (U.S. patent application Ser. No. 10/005,429, filed Dec. 3, 2001)), the disclosures of which are herein incorporated by reference. The polynucleotides of the present invention can also be stacked with traits desirable for insect, disease or herbicide resistance (e.g., Bacillus thuringiensis toxic proteins (U.S. Pat. Nos. 5,366,892; 5,747,450; 5,737,514; 5723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene) and glyphosate resistance (EPSPS gene)) and traits desirable for processing or process products such as high oil (e.g., U.S. Pat. No. 6,232,529); modified oils (e.g., fatty acid desaturase genes (U.S. Pat. No. 5,952,544; WO 1994/11516)); modified starches (e.g., ADPG pyrophosphorylases (AGPase), starch synthases (SS), starch branching enzymes (SBE) and starch debranching enzymes (SDBE)) and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present invention with polynucleotides affecting agronomic traits such as male sterility (e.g., see, U.S. Pat. No. 5,583,210), stalk strength, flowering time or transformation technology traits such as cell cycle regulation or gene targeting (e.g., WO 1999/61619; WO 2000/17364; WO 1999/25821), the disclosures of which are herein incorporated by reference.

In one embodiment, sequences of interest improve plant growth and/or crop yields. For example, sequences of interest include agronomically important genes that result in improved primary or lateral root systems. Such genes include, but are not limited to, nutrient/water transporters and growth induces. Examples of such genes, include but are not limited to, maize plasma membrane H⁺-ATPase (MHA2) (Frias, et al., (1996) Plant Cell 8:1533-44); AKT1, a component of the potassium uptake apparatus in Arabidopsis, (Spalding, et al., (1999) J Gen Physiol 113:909-18); RML genes which activate cell division cycle in the root apical cells (Cheng, et al., (1995) Plant Physiol 108:881); maize glutamine synthetase genes (Sukanya, et al., (1994) Plant Mol Biol 26:1935-46) and hemoglobin (Duff, et al., (1997) J. Biol. Chem. 27:16749-16752, Arredondo-Peter, et al., (1997) Plant Physiol. 115:1259-1266; Arredondo-Peter, et al., (1997) Plant Physiol 114:493-500 and references sited therein). The sequence of interest may also be useful in expressing sense nucleotide sequences of genes that that negatively affects root development.

Additional, agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802 and 5,990,389, herein incorporated by reference. Another example is lysine and/or sulfur rich seed protein encoded by the soybean 2S albumin described in U.S. Pat. No. 5,850,016 and the chymotrypsin inhibitor from barley, described in Williamson, et al., (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. patent application Ser. No. 08/740,682, filed Nov. 1, 1996 and WO 1998/20133, the disclosures of which are herein incorporated by reference. Other proteins include methionine-rich plant proteins such as from sunflower seed (Lilley, et al., (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen, et al., (1986) J. Biol. Chem. 261:6279; Kirihara, et al., (1988) Gene 71:359; both of which are herein incorporated by reference) and rice (Musumura, et al., (1989) Plant Mol. Biol. 12:123, herein incorporated by reference). Other agronomically important genes encode latex, Floury 2, growth factors, seed storage factors and transcription factors.

Insect resistance genes may encode resistance to pests that have great yield drag such as rootworm, cutworm, European Corn Borer and the like. Such genes include, for example, Bacillus thuringiensis toxic protein genes (U.S. Pat. Nos. 5,366,892; 5,747,450; 5,736,514; 5,723,756; 5,593,881 and Geiser, et al., (1986) Gene 48:109), and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432 and Mindrinos, et al., (1994) Cell 78:1089), and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene) or other such genes known in the art. The bar gene encodes resistance to the herbicide basta, the nptII gene encodes resistance to the antibiotics kanamycin and geneticin and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802 and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as 13-Ketothiolase, PHBase (polyhydroxyburyrate synthase) and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones and the like. The level of proteins, particularly modified proteins having improved amino acid distribution to improve the nutrient value of the plant, can be increased. This is achieved by the expression of such proteins having enhanced amino acid content.

This invention can be better understood by reference to the following non-limiting examples. It will be appreciated by those skilled in the art that other embodiments of the invention may be practiced without departing from the spirit and the scope of the invention as herein disclosed and claimed.

### EXAMPLES

### Example 1

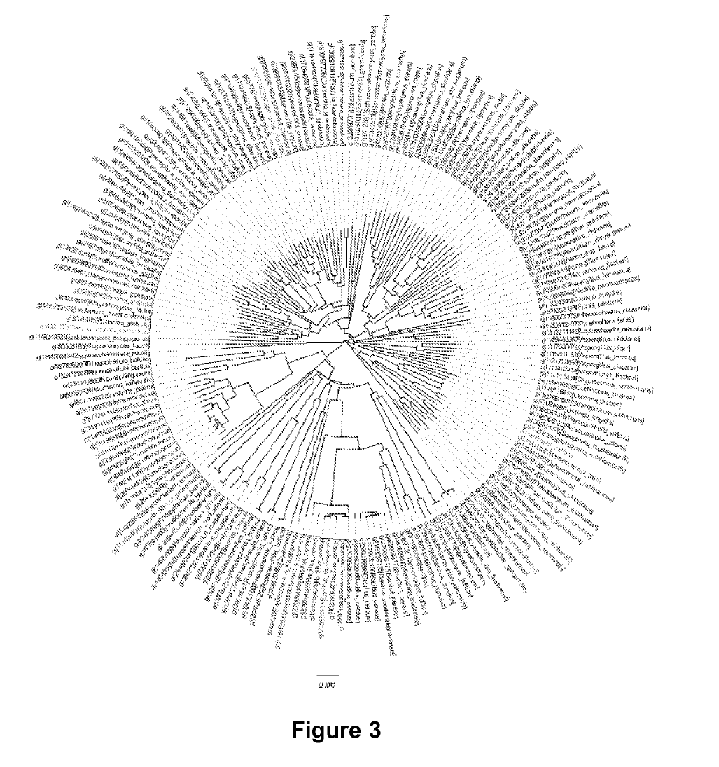

Identification and phylogenetic analysis of potential urea transporter from maize and from diverse microbial and lower plant sources Proteins of several classes which possess the ability to translocate urea across biological membranes have been identified in a variety of species from bacteria to higher eukaryotes such as plants. Using a bioinformatic approach a number of genes were identified encoding potential homologues of the eukaryotic DUR3 high affinity urea transporter (SEQ ID NOS: 1-187), the prokaryotic UREI urea channel (SEQ ID NOS: 188-193), and the aquaporin-like NIP/TIP/PIP proteins (SEQ ID NOS: 194-223). Sequences of potential DUR3 homologues were obtained from a variety of sources, principally focused on microbes, fungi and lower photosynthetic plants and phylogenetic analysis was performed to examine the relationship among transporters (FIG. 3). The probable maize Dur3 homologue was also identified based on sequence homology to characterized urea transporters such as the Arabidopsis thaliana and Saccharomyces cerevisiae Dur3 proteins (Table 2)

TABLE 2

| | Arabidopsis | | |
|---|---|---|---|
| | thaliana | Zea mays | Saccharomyces |
| Species/Protein | Dur3 | Dur3 | cerevisiae Dur3 |
| Arabidopsis thaliana Dur3 | 1 | | |
| Zea mays Dur3 | 0.72 | 1 | |
| Saccharomyces cerevisiae | 0.35 | 0.34 | 1 |

| | |
|---|---|
| Dur3 | Percent Identity |

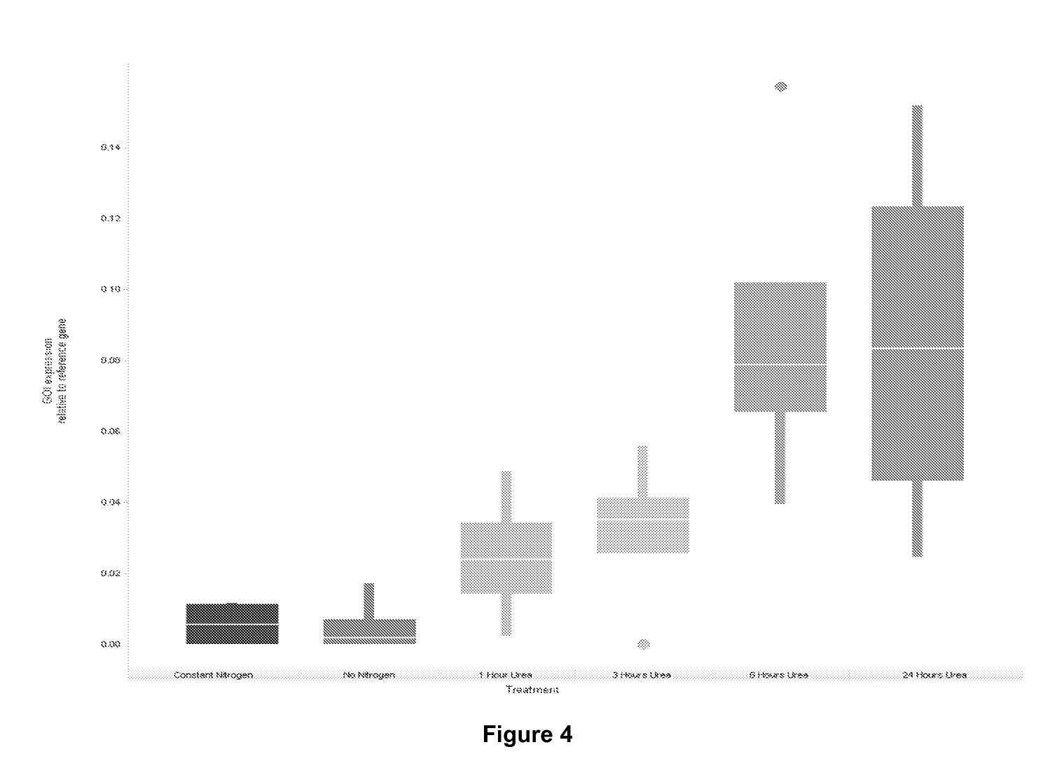

As would be expected for a transporter involved in urea import, qPCR analysis of root tissue from maize seedlings shows the ZmDur3 is induced by switching to growth media containing 10 mM urea as the sole nitrogen source when compared to plants grown in either no nitrogen or grown in constant amounts of nitrate (FIG. 4).

Genes encoding proteins highly similar to the Helicobacter pylori UREI urea channel were also identified using BLAST searches of publicly available databases, as this gene has previously been demonstrated to transport urea with good efficacy. A list of potential UREI homologues based on percent identity to the Helicobacter pylori Urel protein was also generated for subsequent testing (SEQ ID NOS: 190-193 Table 3).

TABLE 3

| | | | % Identity to |
|---|---|---|---|
| Organism | Description | Gene Annotation | HpUREI |
| Streptococcus_thermophilus | Lactic acid bacterium used | Urea channel | 58 |
| | for commercial purposes | | |
| | including production of milk | | |
| | and cheese; not pathogenic | | |
| Lactobacillus_ruminis | Lactic acid bacteriyum | AmiS/UreI transporter | 55 |
| | found normally in the | | |
| | intestine of humans and | | |
| | animals; not pathogenic and | | |
| | considered “probiotic” | | |
| Oxalobacter_formigenes | Oxalate-degrading | Urease accessory | 52 |
| | anaerobic bacterium found | protein UreI | |
| | in the large intestine of | | |
| | animals; not pathogenic and | | |
| | considered “probiotic” | | |
| Desulfotomaculum_nigrificans | Sulfate reducing anaerobic | AmiS/UreI transporter | 43 |
| | bacterium; found in soil and | | |
| | extreme environments; not | | |
| | pathogenic | | |

It has previously been demonstrated that a number of genes belonging to the aquaporin superfamily of channel proteins, the NIPs, TIPs and MIPs, possess the ability to transport urea across biological membranes. These relatively low affinity urea transporters are believed to be localized to both the plasma membrane of cells as well as to the membranes of internal storage structures such as the tonoplast and are possibly involved in urea uptake and urea release from storage sinks. Genes encoding proteins highly similar to the NIP, TIP and MIP low affinity urea channels were also identified using BLAST searches of proprietary and publicly available databases. A list of proteins previously shown to transport urea as well as potential homologues (SEQ ID NOS: 194-223) based on percent identity to the maize orthologues has also been generated for testing in yeast complementation assays as well as for creation of transgenic plants.

### Example 2

### Identification of Potential Urease Genes from Various Plant and Microbial Sources

Urease is an enzyme that is essential for the breakdown of urea into ammonia which can be utilized by the cell. Through bioinformatic approaches a number of genes encoding potential urease proteins have been identified (SEQ ID NOS: 251-298). Analysis focused primarily on single sub-unit urease proteins which are encoded by only one transcript, and sequences of forty-eight known or putative ureases were obtained from a variety of sources, primarily of fungal or plant origin. Phylogenetic analysis was performed to examine the relationship among the known and putative urease proteins (FIG. 4). While expression of a urease protein alone might be sufficient to enhance urease activity in the plant, it is possible that the co-expression of a urease with its corresponding urease accessory proteins might enhance this enzymatic activity. In bacteria, these proteins are easily identifiable as they fall within the same operon as the urease protein, and they are believed to be involved in proper folding, localization, and metal incorporation into the urease enzyme.

### Example 3

### Identification of Potential GS Genes from Maize and Other Sources

Once urea has been broken down into its component parts by urease, released ammonia is assimilated into amino acids, primarily through the action of glutamine synthetase enzymes. Through bioinformatic approaches a number of genes encoding potential glutamine synthetase proteins have been previously identified (see, U.S. patent application Ser. No. 12/607,089, published May 6, 2010). Sequences of two known glutamine synthetases, the GS1-3 isoforms from both Zea mays and from Arabidopsis thaliana are included in this application for incorporation into transgenic plants. While these two GS proteins have been used here, this does not exclude the use of either GS1, GS2 or GS3 type glutamine synthetase proteins from plant or other sources to create transgenics with increased ability to utilize urea as an N source.

### Example 4

### Complementation Assays Using a Yeast dur3A Mutant to Test the Urea Transport Ability In Vivo of Potential Urea Transport Proteins

Genes encoding a number of the previously listed potential DUR3 homologues selected from diverse evolutionary clades (labeled in blue in FIG. 3), as well as the known urea transporters Arabidopsis thaliana DUR3 and Helicobacter pylori UREI, were synthesized into the yeast expression vector pJR1133. The vectors were transformed into a dur3A yeast strain which has a targeted deletion of the gene encoding its high affinity urea transport protein and the transgenes were expressed from the constitutive GPD promoter. The ability of each protein to transport urea was measured by assaying each for its ability to complement the dur3A growth phenotype on minimal media supplemented with 1 mM to 20 mM urea as the sole nitrogen source. The average results of at least two growth complementation assays as well as a qualitative assessment of the function of each protein as a urea transporter are listed in Table 4 below. For qualitative scoring “very good” corresponds to complementation at less than 1 mM urea, “good” corresponds to complementation at 1-3 mM urea, “fair” corresponds to complementation at 3-5 mM urea, “poor” corresponds to complementation at 5-10 mM urea, and “none” corresponds to no complementation evident at concentrations above 10 mM urea.

TABLE 4

| | Score (avg. of 2 | [Urea] Supporting | | Used in |
|---|---|---|---|---|
| Transporter | assays) | Growth | | Bioinformatics? |
| Wild-Type/Vector | Very Good | <1 | mM | No |
| dur3Δ/Vector | None | >10 | mM | No |
| dur3Δ/Arabidopsis DUR3 | Fair | 3-5 | mM | Yes: Functional |
| dur3Δ/Maize DUR3 | Fair | 3-5 | mM | No |
| dur3Δ/Micromonas | Fair | 5 | mM | No |
| DUR3 | | | | |
| dur3Δ/Thalassiosira | Fair | 3-5 | mM | No |
| DUR3 | | | | |
| dur3Δ/Selaginalla DUR3 | Good | 2-3 | mM | Yes: Functional |
| dur3Δ/Synechococcus | Fair | 3-5 | mM | Yes: Functional |
| DUR3 | | | | |
| dur3Δ/Ostreococcus | Fair | 3-5 | mM | No |
| DUR3 | | | | |
| dur3Δ/Bacillus | Fair | 5 | mM | No |
| pseodofirmus DUR3 | | | | |
| dur3Δ/Idiomarina DUR3 | Poor | 7.5 | mM | No |
| dur3Δ/Mycobacterium | Poor/None | >10 | mM | Yes: Non-Functional |
| DUR3 | | | | |
| dur3Δ/Debaryomyces | Fair | 5 | mM | Yes: Functional |
| DUR3 | | | | |
| dur3Δ/Pichia DUR3 | Fair | 3-5 | mM | Yes: Functional |
| dur3Δ/Geobacillus DUR3 | Poor/None | >10 | mM | Yes: Non-Functional |
| dur3Δ/Bacillus | Fair | 5 | mM | No |
| thuringiensis DUR3 | | | | |
| dur3Δ/Chlamydomonas | Fair | 5 | mM | No |
| DUR3 | | | | |
| dur3Δ/Phaeodactylum | Fair | 3-5 | mM | Yes: Functional |
| DUR3 | | | | |
| dur3Δ/Helicobacter UREI | Good | 1-2 | mM | No |
| dur3Δ/Actinobacillus | Poor | >10 | mM | No |
| UREI | | | | |
| dur3Δ/Desulfotomaculum | Fair | 3-4 | mM | No |
| UREI | | | | |
| dur3Δ/Lactobacillus | Good | 1-2 | mM | No |
| UREI | | | | |
| dur3Δ/Oxalobacter UREI | Fair | 4-5 | mM | No |
| dur3Δ/Streptococcus | Good | 5 | mM | No |
| UREI | | | | |

Transporters included in subsequent bioinformatics analyses as examples of verified urea transporters (verified experimentally or ascertained from literature review) are further assigned the value “Yes: Functional” in the last column, and transporters selected as having little or no ability to transport urea are assigned the value “Yes: Non-Functional”.

While screening for urea transporters was performed using growth assays of dur3A yeast cells which are defective in urea transport, other methods of screening known in the art could also be utilized. This includes but is not limited to screening for growth on urea as a nitrogen source using other genetically pliable organisms such as bacteria or cultured mammalian cells. Screening for uptake of urea, possibly radioactively labeled, in any of a number of systems including yeast, bacteria, cultured mammalian cells, Xenopus oocytes, membrane vesicle or any equivalent manner.

### Example 5

### Bioinformatics Approach to Define Domains, Motifs, and Residues Critical for the Ability of DUR3 Homologues to Transport Urea Across Cellular Membranes

Initial screening in yeast identified several Dur3-type proteins with the ability to transport urea into the cell, and sequence analysis of these proteins as well as other previously characterized was performed to residues and domains involved in urea transport. This knowledge was then applied in more stringent database analyses to discover proteins with an improved capacity for urea transport. The MOTIFS program of the Blocks server found on the world wide web at blocks.fhcrc.org was used to create alignments and to identify conserved block regions from various DUR3 homologues either previously demonstrated in the published literature or identified in screening as capable of transporting urea across a biological membrane. Protein sequences of DUR3 homologues from Arabidopsis thaliana, Aspergillus nidulans, Debaromyces hansenii, Oryza sativa-Japonica, Phaeodactylum tricornutum, Pichia angusta, Saccharomyces cerevisiae, Selaginella moellendorffii and Synechococcus sp.WH7805 were used to create alignments and identify conserved blocks (Table 5).

TABLE 5

| | Amino Acid | | |
|---|---|---|---|
| | Positions | | |
| | modeled from | | SEQ |
| | Selaginella | Amino Acid | ID |
| Block | moellendorffii | Sequence | NO: |
| 1 | aa 116-aa167 | PFWYASGATIQVLLFGII | 310 |
| | | AIEIKRKAPSAHTVCEIV | |
| | | RARWGFEAHMVFLSFC | |
| 2 | aa 189-aa240 | LTGVDIYAASFLIPLGVI | 311 |
| | | VYTLAGGLKATFLASYIH | |
| | | SVIVHVVLVIFVYLVY | |
| 3 | aa 279-aa318 | PVSGNYGGSYVTMLSSGG | 312 |
| | | LVFGIINIVGNFGTVFVD | |
| | | NGYW | |
| 4 | aa 374-aa427 | PATATALMGNSGAILLLT | 313 |
| | | MLFMAVTSAGSSELVAVS | |
| | | SLCTYDIYRTYINPKATG | |
| 5 | aa 456-aa498 | RVSLGWMYLAMGVMVGSA | 314 |
| | | VMPIAFLLLWSKANAKGA | |
| | | IAGTIVG | |

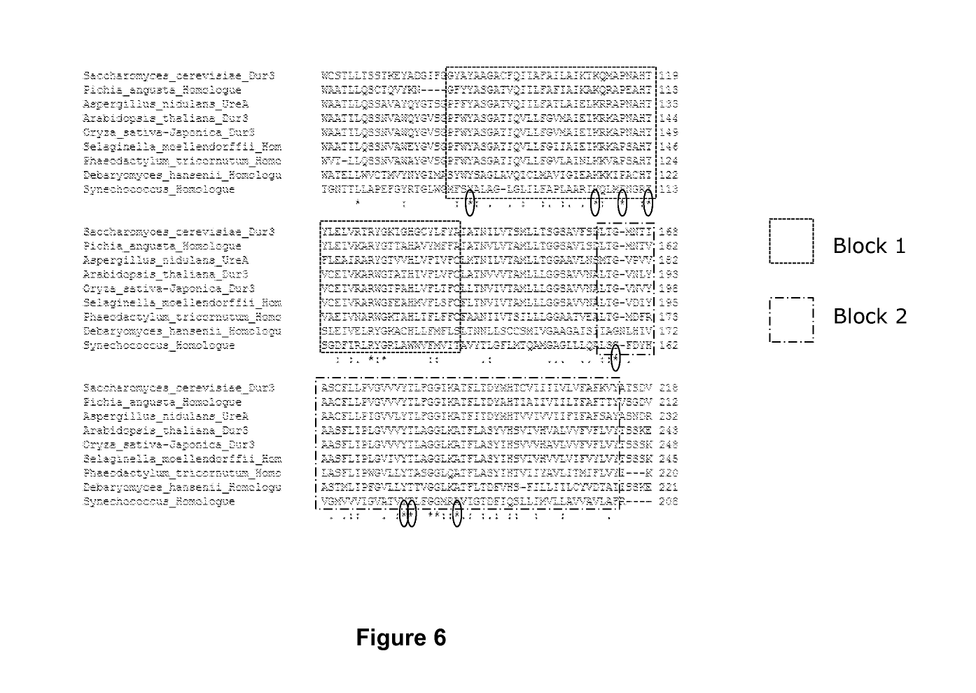

These blocks were modeled on alignment files created using the ClustalW2 multiple sequence alignment program and amino acid residues that were absolutely conserved among all of the sequences initially used to define the blocks were identified. Using these conserved residues as a template, a second in silico screen to identify the subset of amino acid residues which are not conserved in either the Geobacillus kaustophilus or the Mycobacterium smegmatis sequence (both demonstrate little or no ability to transport urea in complementation assay) was performed. The residues absolutely conserved in verified transporters and not conserved in either of two non-functional urea transporters were used to create “urea-critical” motifs which are predicted to have a major positive influence on the ability to transport urea in vivo. An example of this analysis for Blocks 1 and 2 is shown in FIG. 6 with an alignment of the transporters shown in a Clustal W format and the residues used to define the urea critical motif which are conserved in functional transporters and not conserved in at least one non-functional transporter circled. The six urea critical motifs identified are presented in this application (SEQ ID NOS). Urea-critical motifs were used to re-analyse the original list of putative DUR3-type urea transporters presented in FIG. 3, and proteins which were conserved at all of these residues were selected for synthesis and further screening (SEQ ID NOS: 315-321).

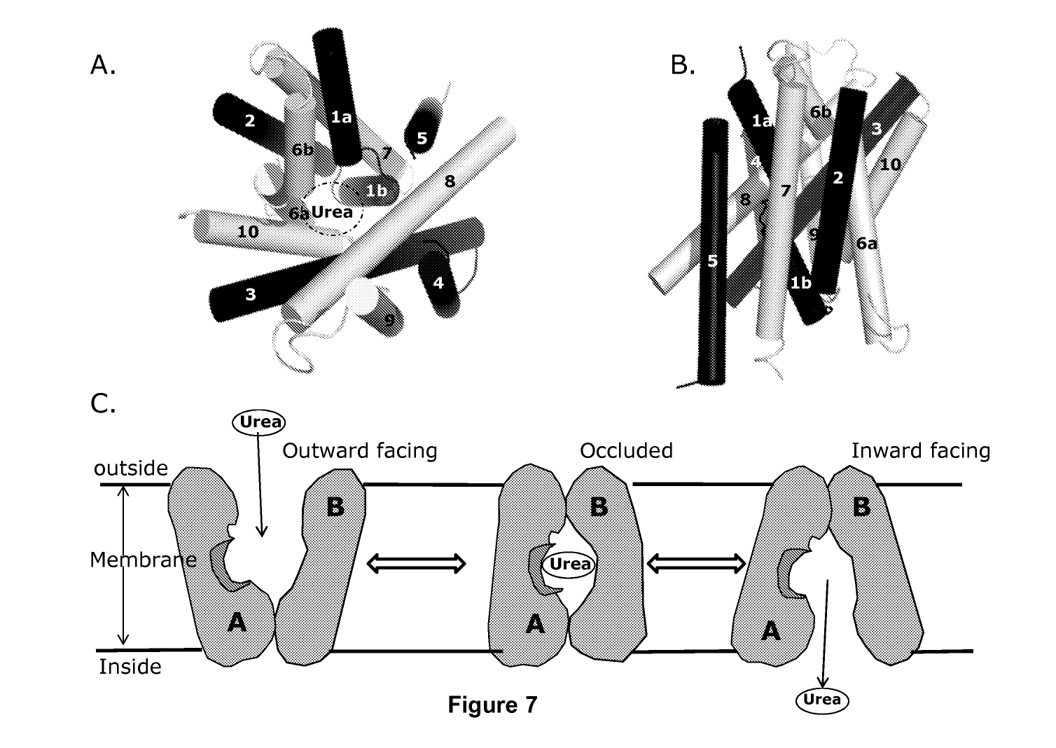

X-ray crystallography demonstrates that the SSF family of proteins shares a remarkably conserved transporter core containing an inverted repeat of 5 transmembrane (TM) helices (FIGS. 7A and 7B). The pseudo 2-fold symmetric relationship of TMs 1-5 and 6-10 enables the transporter to undergo conformation recycling from outward-facing to occluded and finally to inward-facing, a typical rocket-switch or alternative-access mechanism with a single substrate binding site at the center of transporter (FIG. 7C). Another unusual but conserved feature among these transporter structures is that the TM1 helix and its symmetrical counterpart TM6 are unwound or “kinked” in the middle of the transmembrane domain. Consequently, the broken helices provide ample backbone carbonyl and amide groups to facilitate recognition of the incoming substrate through hydrogen bonds. These coiled residues in TM1 and TM6 combined with the middle sections of TM3 and TM8 make up the substrate binding site.

Among proteins with known 3D structures, the Saccharomyces cerevisiae Dur3 (Sc_Dur3) sequence is best matched to sodium/galactose transporter from Vibrio parahaemolyticus (vSGLT, PDB:2xq2, ref 2) with a marginal sequence identity of ˜22%. To verify this match's significance, PSI-Blast (Position-Specific-Iterative or profile Blast) was employed to search a new data set which contains swiss-prot and SSF transporter sequences with known structures. The Sc_Dur3 and vSGLT alignment in the second round search produces a significant e value of ˜1e-69. TMHMM and other transmembrane helix prediction tools suggest Sc-Dur3 has15 transmembrane helices, more than enough to form the symporter core domain, with extra helices likely forming periphery structure not essential to transport activity but possibly playing a functional role in regulation of the protein. Using the sequence/structure alignment and TM helix prediction, we have identified the 10 core transmembrane helices and the putative urea binding sequence motifs. A 3D membrane imbedded topology of the 10 TM core domain was also constructed by threading Sc-Dur3 sequence through vSGLT structure (FIG. 7). The model reveals that the putative binding site is at the center of protein, suggesting protein motifs of middle sections of TM1, TM3, TM6 and TM8 are important for urea recognition while TM2, TM4, TM5, TM7, TM9 and TM10 likely play in a structural role forming a scaffold to enable confirmation recycling during urea uptake. The approximate location of these transmembrane domains, modeled on the Penicillium Dur3 protein, are defined in Table 6. All fifteen TM helices are defined as well as the ten core transmembrane domains (labeled cTM1-cTM10). Proposed function of each transmembrane domain is also given in the table.

TABLE 6

| Transmembrane | Correlation | | |
|---|---|---|---|
| Domain (Predicted | to Core | Amino Acid | |
| using HMMTOP) | Structure | Residues | Presumed Function |
| TM1 | −1 | aa30-aa49 | Peripheral |
| TM2 | cTM 1 | aa62-aa86 | Central Pore Forming; |
| | | | Probable Urea |
| | | | Binding Site |
| TM3 | cTM 2 | aa95-aa114 | Structural |
| TM4 | cTM 3 | aa139-aa163 | Central Pore Forming; |
| | | | Probable Urea |
| | | | Binding Site |
| TM5 | cTM 4 | aa172-aa191 | Structural |
| TM6 | cTM 5 | aa200-aa219 | Structural |
| TM7 | cTM 6 | aa258-aa275 | Central Pore Forming; |
| | | | Probable Urea |
| | | | Binding Site |
| TM8 | cTM 7 | aa296-aa320 | Structural |
| TM9 | cTM 8 | aa351-aa375 | Central Pore Forming; |
| | | | Probable Urea |
| | | | Binding Site |
| TM10 | cTM 9 | aa400-aa419 | Structural |
| TM11 | cTM 10 | aa428-aa447 | Structural |
| TM12 | +1 | aa454-aa473 | Peripheral |
| TM13 | +2 | aa492-aa515 | Peripheral |
| TM14 | +3 | aa573-aa597 | Peripheral |
| TM15 | +4 | aa606-aa625 | Peripheral |

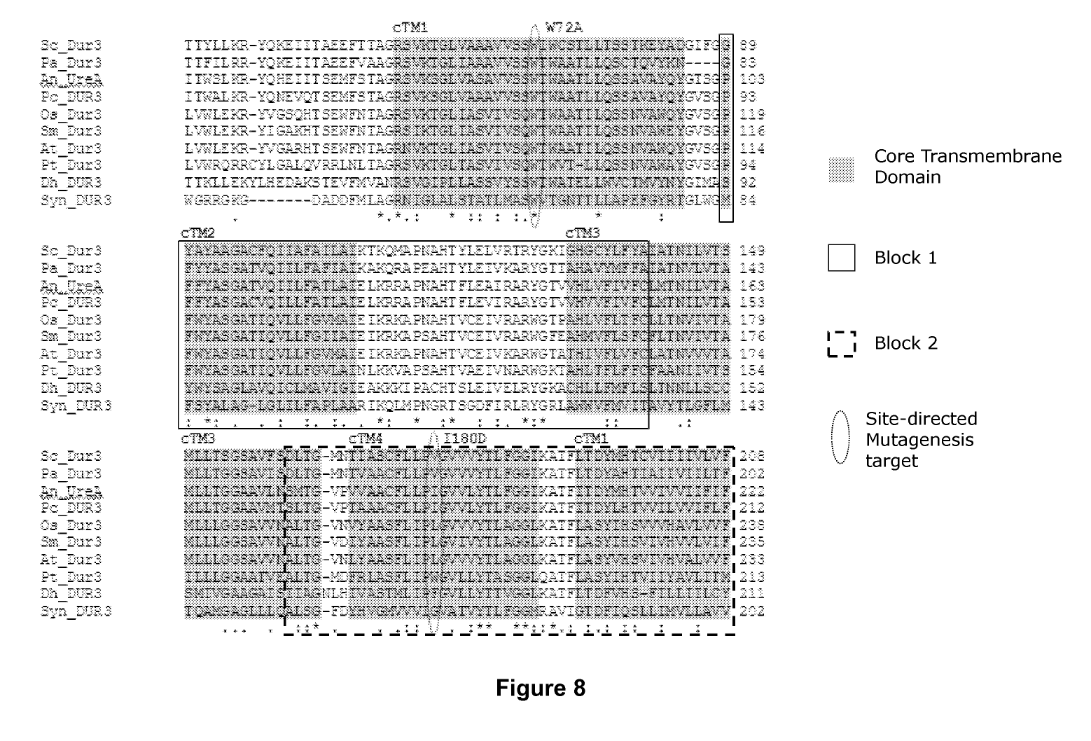

The validity of this model is also supported by site mutagenesis on Penicillium chrysogenum Dur3 as shown in FIG. 8 and Table 6. The I180 residue is located in the middle of cTM4 and likely interacts with the plasma membrane. Among verified Dur3-type urea transporters this position is conserved and occupied by hydrophobic residues such as I, L and V (marked by dashed oval in FIG. 8). Mutation of I180 to aspartic acid (D) introduces a hydrophilic charged residue which should drastically disrupt the structural integrity of the transporter. Consistent with this hypothesis the I180->D mutant shows clear defects in its ability to transport urea (Table 7). W72 (marked by dashed oval in FIG. 8) is near the unwound area of cTM1 and is likely involved in urea binding. Mutation of W72 to Alanine (A) results in a moderate inability to take urea into the cell. Both mutated residues fall in or near previously described conserved Blocks 1 and 2 in the N-terminal half of the urea transporter. The first block covers a protein motif between TM2 and TM3. It has been shown that this interhelical motif forms a helix and inserts itself into the central substrate binding cavity from the extracellular side, likely serving as an entrance gate modulating substrate access. The second block mainly consists of TM4 and TM5 (FIG. 8). Based on the 3D model it likely plays a structural role and supports the transporter's conformational flexibility.

TABLE 7

| Mutation Type | Amino Acid Change | Effect on Urea Uptake |
|---|---|---|
| Urea Binding Site | W (72) → A | Moderate |
| Structural | I (180) → D | Severe |

### Example 6

### Testing of Novel Urea Transporters for the Ability to Transport Low Levels of Urea with High Affinity

As levels of urea in the soil are dramatically decreased by the breakdown of the fertilizer by microbes and soil resident urease proteins, the ability to efficiently compete for low levels of urea will be critical under certain conditions. Six additional novel urea transporters similar to the high affinity DUR3 transporter and having complete conservation of all urea-critical residues identified through bioinformatics have been synthesized and tested for the ability to complement a dur3A yeast mutant. The Oryza DUR3 has been included in this assay as a positive control, as it has previously been demonstrated to rescue yeast as well as Arabidopsis DUR3 deletion mutants. A summary of results from multiple complementation assays are shown below in Table 8, and qualitative scores are similar to those described in Example 4.

TABLE 8

| | | |
|---|---|---|
| | | [Urea] |
| | | Supporting |
| Transporter | Score (avg. of 2 assays) | Growth |

| | | | |
|---|---|---|---|
| Wild-Type/Vector | Very Good | <1 | mM |
| dur3Δ/Vector | None | >10 | mM |
| dur3Δ/Ashbya DUR3 | Very Good | <1 | mM |
| dur3Δ/Lachancea DUR3 | Very Good | <1 | mM |
| dur3Δ/Oryza DUR3 | Fair | 4-5 | mM |
| dur3Δ/Penicillium DUR3 | Very Good | <1 | mM |
| dur3Δ/Physcomitrella DUR3 | Poor/None | >10 | mM |
| dur3Δ/Podospora DUR3 | Very Good | <1 | mM |
| dur3Δ/Tuber DUR3 | Good | 1-2 | mM |

Several potential urea transporters which tested positive in yeast growth assays for complementation of dur3A phenotype were subsequently screened for the ability to mediate urea uptake at relatively low substrate levels of less than or equal to 500 μM. Yeast growth assays were performed similar to previously described in media supplemented with between 50 μM and 500 μM urea as the sole nitrogen source, and a summary of results with the lowest amount of urea supporting growth among the concentrations tested is shown in Table 9 below.

TABLE 9

| | Minimal [Urea] Concentration Supporting |
|---|---|
| Transporter | Growth |
| dur3Δ/Lachancea DUR3 | 50 μM |
| dur3Δ/Penicillium DUR3 | 50 μM |
| dur3Δ/Podospora DUR3 | 50 μM |
| dur3Δ/Selaginalla DUR3 | 50 μM |
| dur3Δ/Tuber DUR3 | 50 μM |
| dur3Δ/Lactobacillus UREI | 500 μM |

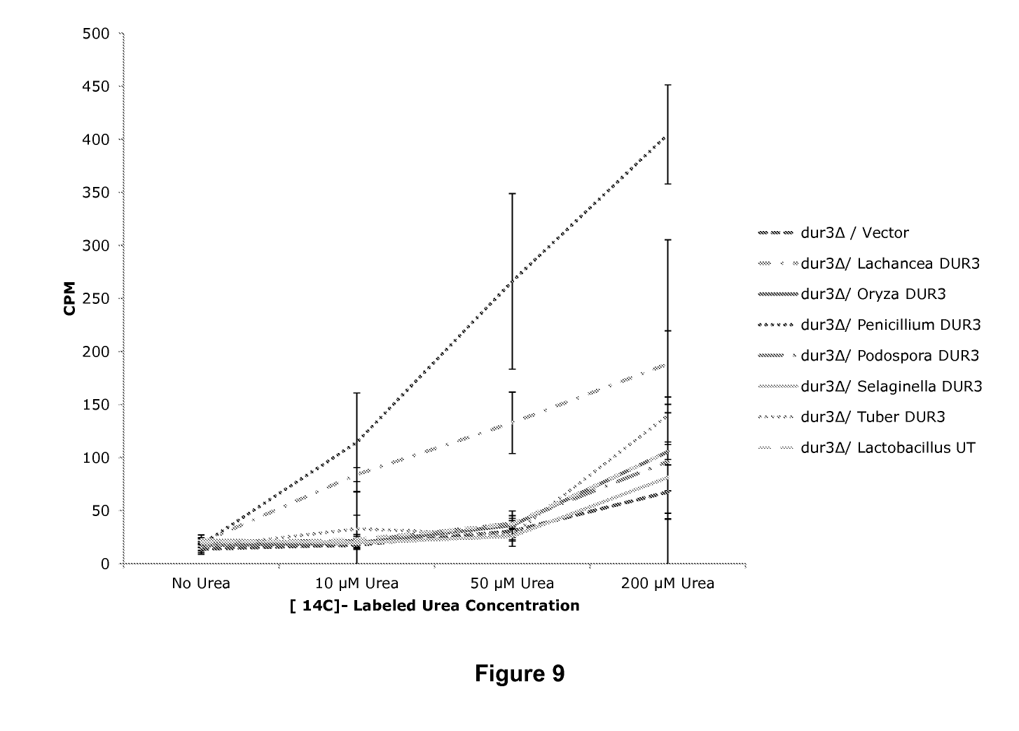

Uptake assays of [¹⁴C]-urea into dur3A yeast cells expressing one of several urea transporters identified during screening were performed essentially as previously described with slight modifications (Morel, et al., Fungal Genetics and Biology, 2008). As seen in FIG. 9, many of the transporters dramatically increased the amount of labeled urea taken into the cell even at extracellular concentrations as low as 10 μM and at relatively short times of 4 minutes.

### Example 7

### Expression/Phenotype of Arabidopsis and Maize Transgenic Plants

The model organism Arabidopsis thaliana was used to test the ability of non-native urea transporters to function in plants. Vectors containing various promoters including the endogenous AtDur3 (SEQ ID NO: 303) promoter or the constitutive 35S promoter (SEQ ID NO: 304) driving expression of various urea transporters identified in yeast screening have been created using polynucleotides optimized for expression in Arabidopsis Wild type Arabidopsis (Columbia-0) have been transformed with these expression vectors by an Agrobacterium mediated ‘floral-dip’ method, and several transgenic lines have been identified by selecting the T₀ seeds for herbicide resistance in soil. Molecular characterization of RNA preparations from these transgenic lines has been performed by qPCR to determine expression of each transgene relative to the elF4g control gene and several lines with significant levels of transgene expression have been identified after molecular analysis. Experiments to express the various urea transporters earlier identified are to be performed. Constructs designed for transgene expression from the ZmUBI, ZmRM2 or ZmRCC3 promoter (SEQ ID NOS: 308, 306 and 309) have been produced and transformed into Gaspe germplasm using a previously established Agrobacterium mediated transformation protocol. Transgene expression from Tₒ will be used to select events to be assessed for general growth and reproductive parameters as well as screened for an increased ability to use urea as a nitrogen source.

To test the effect of transgene expression on Arabidopsis plant performance during growth on urea as a nitrogen source, plants are to be grown in an agarose based sterile soilless system and supplemented with half strength salts as described by Murashige and Skoog, Gambourg's vitamin mix, NiSO₄ to a final concentration of 1 uM and urea at various concentrations between 0.1 μM and 20 μM. T₀ plants which are expressing the transgene have been selected by the presence of the yellow fluorescent protein (YFP), also encoded by the transformation vector, and seedlings not expressing YFP are used as a null control in further experiments. T₁ plants are generated, and these are analyzed at various timepoints post plating on the previously described agarose medium for alteration in growth rates by calculating total leaf area and relative green area of leaves, and transgenic mean parameters are compared to corresponding mean parameters of non-transgenic null controls. After comparison of various growth parameters, plants are harvested and root and shoot total dry weights are determined (after separating the parts and drying at 70° C. for 70 hours). The dried tissue will also be ground and total reduced N is to be measured by the micro-Kjeldahl method. Transgenic mean parameters will be compared to mean parameters of non-transgenic controls.

To test efficacy of transgene expression on plant performance in maize, urea uptake by the transgenic plants or root sections from these transgenics will be assessed by monitoring uptake of [¹⁴—C]-labeled urea and/or [¹⁵—N]-labeled urea similar to previously describe). For growth assays plants will be grown either hydroponically or in a soilless turface based substrate in modified Hoagland's solution with urea supplemented as the sole source of nitrogen. Root and shoot total dry weights (after separating the parts and drying at 70° C. for 70 hours) of plants grown with urea as a nitrogen source will also be calculated and the dried tissue will be ground and total reduced N is to be measured by the micro-Kjeldahl method as well. Transgenic mean parameters will be compared to mean parameters of non-transgenic controls.

### Example 8

### Transgenic Expression of a Urea Transporter, a Urease and a Glutamine Synthetase Using a Single Three Cassette Vector

As use of urea by plant cells could be limited by its import into the cell, by its breakdown in the cell, or by the assimilation of its breakdown products into usable forms, stacking of genes involved in any combination of these processes will be used in transgenic plants. Experiments to test the efficacy of co-expression of the various urea transporters along with a urease gene to catabolize urea and a glutamine synthetase to assimilate the liberated ammonia (transgenic stacks) are to be performed. Constructs designed for transporter expression from the root preferred ZmRM2 promoter (SEQ ID NO: 306) with ZmUrease and ZmGS1-3 expressed from the leaf preferred ZmPEPC promoter (SEQ ID NO: 305) have been created and transformed into elite maize germplasm. Constructs for constitutive expression of the various transporters along with AtUrease and AtGS1-3 expressed from the 35S (SEQ ID NO: 304) promoter have also been constructed and transformed into Arabidopsis using an Agrobacterium mediated transformation protocol. While these are specific examples of genetic stacks engineered in plants, any promoter/gene combination of urea transporter/channel with a urease gene and a glutamine synthetase gene could also be used. Table 10 gives a list of a few possible gene/promoter combinations that could also be employed.

TABLE 10

| Transgene | Promoter Type |
|---|---|
| High Affinity Urea | Constitutive (High, Medium, or Low) |
| Transporter | |
| Low Affinity Urea Channel | Green Tissue Specific |
| Urease | Root Preferred (Stele, Cortex, or Nitrate |
| | Inducible) |
| Glutamine Synthetase | Urea Inducible |
| | Nitrate Inducible |
| | Drought inducible |
| | Diurnally regulated |
| | Guard Cell Preferred |
| | Ear Preferred |
| | Stalk Preferred |
| | Seed Specific (Embryo + aleurone, |
| | Endosperm, or Pericarp) |

For both maize and Arabidopsis, RNA expression analysis of Tₒ events will be used to select events expressing all three transgenes and seeds from these plants are to be assessed for general growth parameters as well as screened for an increased ability to use urea as a nitrogen source. Two of these constructs are also to be tested in elite hybrid corn using field assays for yield. Urea uptake by the transgenic stacks will be assessed by monitoring uptake of [¹⁴—C]-labeled urea and/or [¹⁵—N]-labeled urea. Root and shoot total dry weights (after separating the parts and drying at 70° C. for 70 hours) of plants grown with urea as a nitrogen source will be calculated and the dried tissue will also be ground and total reduced N is to be measured by the micro-Kjeldahl method. Transgenic mean parameters will be compared to mean parameters of non-transgenic controls. Arabidopsis transgenics are also to be assessed for ability to grow on urea as a nitrogen source as described in Example 7. Urease activity of transgenic stacks will be assessed using a modified protocol similar to what has previously been reported by Witte and Medina-Escobar, Analytical Biochemistry, 2001 or an equivalent method. Glutamine synthetase assays are to be performed as previously reported by Kingdon, et al, Biochemistry, 1968 or an equivalent method. In all cases, transgenic mean parameters will be compared to mean parameters of either wild-type plants or non-transgenic null controls.

### Example 9

### Overexpression of Genes Involved in Urea Uptake and Assimilation to Enhance Foliar Uptake of Urea

Using the previously described maize Gaspe transgenics expressing either a urea transporter alone or a transporter stacked with a urease and glutamine synthetase, plants are to be tested for an increased ability to uptake [¹⁵N]-labeled urea applied to leaves as an aqueous solution. This will be done using a method similar to what has previously described by Below, et al, Agronomy Journal, 1985. After foliar application of the labeled urea, various tissues will be harvested and the uptake and incorporation of N will be examined by determining the concentration of ¹⁵N in each tissue using mass spectrometry. Uptake and incorporation of ¹⁵N by transgenic plants will be assessed relative to null plants coordinately treated with equal amounts of labeled urea. Furthermore, total nitrogen levels as well as levels of nitrate and free amino acids will also be determined as these parameters have previously been demonstrated to increase after foliar application of urea.

### Example 10

### Stacking and Co-Expression of a High Affinity and a Low Affinity Urea Transporter to Increase Urea Uptake Across a Range of Extracellular Urea Concentrations

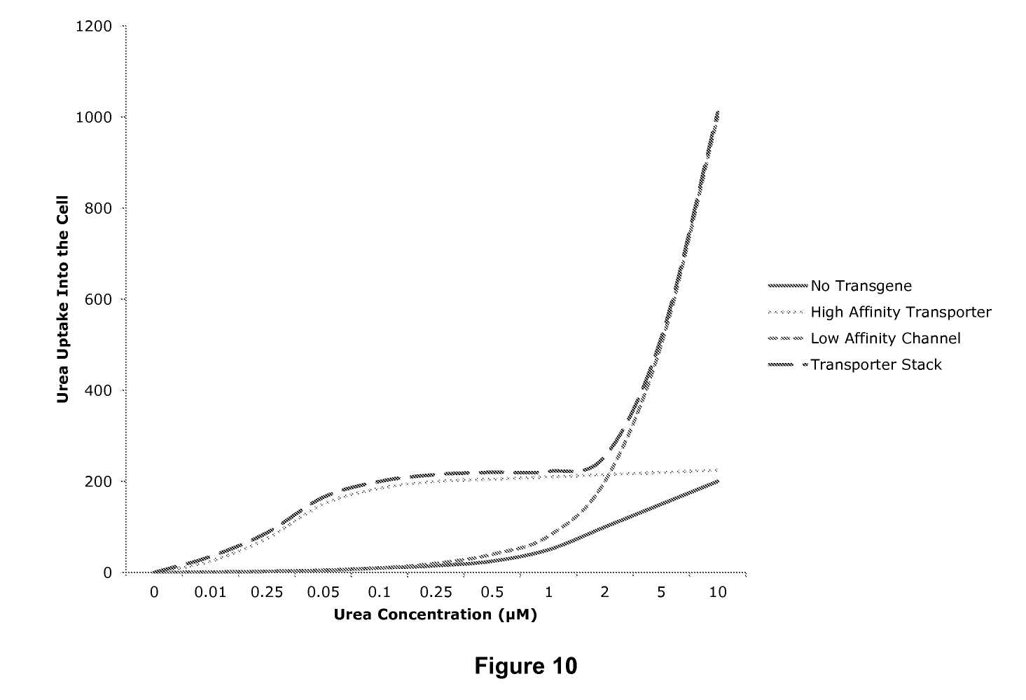

In modern agronomic practices, urea fertilizer is often applied to a crop at a limited number of times during a growing cycle. This leads to urea concentrations in the soil that can vary by orders of magnitude depending on when urea was applied and what the prevailing environmental conditions have been in the interim. Co-expression of a high affinity transporter capable of extracting very scarce urea from the soil and a low affinity channel which could increase uptake of urea when it is available in excess should increase uptake over a wider range of concentrations allowing efficient uptake of urea by transgenics regardless of the concentration of the substrate in the soil. Examples of newly identified proteins similar to both the known Dur3-type high affinity urea transporters as well as the lower affinity Urel-type urea channels have been presented previously in this patent. Experiments to test the efficacy of co-expression of multiple urea transporters from both the Dur3 family of high affinity urea transporters as well as from the Urel family of urea channel proteins are in progress, however similar co-expression stacks could be employed with any combination of transporters with differing Kₘ and Vₘₐₓ values. Constructs designed for co-expression of two urea transporters from both a constitutive and a root preferred promoter have been designed and created (Table 11). These constructs are to be transformed into both Arabidopsis and maize Gaspe germplasm using a previously established Agrobacterium mediated transformation protocol. Positive transgene events will be assessed for general growth and reproductive parameters as well as screened for an increased ability to use urea as a nitrogen source. Transgenic plants will be assessed for the potential of enhanced urea uptake at both low and high levels of extracellular urea, and significant events would be expected to increase uptake and N status of the cell over a wide range of urea in the growth medium as illustrated in FIG. 10.

TABLE 11

| High Affinity | | Low Affinity | | |
|---|---|---|---|---|
| Transporter | Promoter | Channel | Promoter | Plant Host |
| Selaginella DUR3 | 35S (Constitutive) | Helicobacter UREI | 35S (Constitutive) | Arabidopsis |
| Selaginella DUR3 | 35S (Constitutive) | Lactobacillus UREI | 35S (Constitutive) | Arabidopsis |
| Lachancea DUR3 | 35S (Constitutive) | Helicobacter UREI | 35S (Constitutive) | Arabidopsis |
| Lachancea DUR3 | 35S (Constitutive) | Lactobacillus UREI | 35S (Constitutive) | Arabidopsis |
| Penicillium DUR3 | 35S (Constitutive) | Helicobacter UREI | 35S (Constitutive) | Arabidopsis |
| Penicillium DUR3 | 35S (Constitutive) | Lactobacillus UREI | 35S (Constitutive) | Arabidopsis |
| Podospora DUR3 | 35S (Constitutive) | Helicobacter UREI | 35S (Constitutive) | Arabidopsis |
| Podospora DUR3 | 35S (Constitutive) | Lactobacillus UREI | 35S (Constitutive) | Arabidopsis |
| Tuber DUR3 | 35S (Constitutive) | Helicobacter UREI | 35S (Constitutive) | Arabidopsis |
| Tuber DUR3 | 35S (Constitutive) | Lactobacillus UREI | 35S (Constitutive) | Arabidopsis |

### Example 11

### Transformation and Regeneration of Transgenic Plants by Agrobacterium-Mediated Transformation

For Agrobacterium-mediated transformation of maize with a sense sequence of the urea transporter, urease or glutamine synthetase sequence of the present invention, preferably the method of Zhao is employed (U.S. Pat. No. 5,981,840 and PCT Patent Publication WO 1998/32326, the contents of which are hereby incorporated by reference). Briefly, immature embryos are isolated from maize and the embryos contacted with a suspension of Agrobacterium, where the bacteria are capable of transferring the sense urea transporter, urease, or glutamine synthetase sequences to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step) and preferably calli grown on selective medium are cultured on solid medium to regenerate the plants. Plants are monitored and scored for a modulation in tissue development.

Immature maize embryos from greenhouse donor plants are bombarded with a plasmid containing the urea transporter, urease, or glutamine synthetase sequence operably linked to constitutive or tissue specific promoter (Vilardell, et al., (1990) Plant Mol Biol 14:423-432) and the selectable marker gene PAT, which confers resistance to the herbicide Bialaphos. Alternatively, the selectable marker gene is provided on a separate plasmid. Transformation is performed as follows. Media recipes follow below.

Preparation of Target Tissue:

The ears are husked and surface sterilized in 30% Clorox® bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA:

A plasmid vector comprising the urea transporter, urease, or glutamine synthetase sequence operably linked to an ubiquitin promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in Tris EDTA buffer (1 pg total DNA)

100 μl 2.5 M CaCl₂

10 μl 0.1M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment:

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment:

Following bombardment, the embryos are kept on 560Y medium for 2 days, then transferred to 560R selection medium containing 3 mg/liter Bialaphos and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for increased drought tolerance. Assays to measure improved drought tolerance are routine in the art and include, for example, increased kernel-earring capacity yields under drought conditions when compared to control maize plants under identical environmental conditions. Alternatively, the transformed plants can be monitored for a modulation in meristem development (i.e., a decrease in spikelet formation on the ear). See, for example, Bruce, et al., (2002) Journal of Experimental Botany 53:1-13.

Bombardment and Culture Media:

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D and 2.88 g/l L-proline (brought to volume with D-I H₂O following adjustment to pH 5.8 with KOH); 2.0 g/l Gelrite® (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/I Eriksson's Vitamin Mix (1000×SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite® (added after bringing to volume with D-I H₂O) and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/I MS vitamins stock solution (0.100 g nicotinic acid, 0.02 g/l thiamine HCL, 0.10 g/l pyridoxine HCL and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose and 1.0 ml/I of 0.1 mM abscisic acid (brought to volume with polished D-I H₂O after adjusting to pH 5.6); 3.0 g/l Gelrite® (added after bringing to volume with D-I H₂O) and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/I MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol and 40.0 g/l sucrose (brought to volume with polished D-I H₂O after adjusting pH to 5.6) and 6 g/l Bacto™-agar (added after bringing to volume with polished D-I H₂O), sterilized and cooled to 60° C.

### Example 12

### Soybean Embryo Transformation

Soybean embryos are bombarded with a plasmid containing a sense urea transporter, urease, or glutamine synthetase sequences operably linked to an ubiquitin promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein, et al., (1987) Nature (London) 327:70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell, et al., (1985) Nature 313:810-812), the hygromycin phosphotransferase gene from plasmid pJR225 (from E. coli; Gritz, et al., (1983) Gene 25:179-188) and the 3′ region of the nopaline synthase gene from the T-DNA of the Ti plasmid of Agrobacterium tumefaciens. The expression cassette comprising a sense urea transporter, urease, or glutamine synthetase sequence operably linked to the ubiquitin promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 13

### Sunflower Meristem Tissue Transformation

Sunflower meristem tissues are transformed with an expression cassette containing a sense urea transporter, urease or glutamine synthetase sequence operably linked to a ubiquitin promoter as follows (see also, EP Patent Number 0 486233, herein incorporated by reference and Malone-Schoneberg, et al., (1994) Plant Science 103:199-207). Mature sunflower seed (Helianthus annuus L.) are dehulled using a single wheat-head thresher. Seeds are surface sterilized for 30 minutes in a 20% Clorox® bleach solution with the addition of two drops of Tween® 20 per 50 ml of solution. The seeds are rinsed twice with sterile distilled water.

Split embryonic axis explants are prepared by a modification of procedures described by Schrammeijer, et al., (Schrammeijer, et al., (1990) Plant Cell Rep. 9:55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige, et al., (1962) Physiol. Plant., 15:473-497), Shepard's vitamin additions (Shepard, (1980) in Emergent Techniques for the Genetic Improvement of Crops (University of Minnesota Press, St. Paul, Minn.), 40 mg/l adenine sulfate, 30 WI sucrose, 0.5 mg/l 6-benzyl-aminopurine (BAP), 0.25 mg/l indole-3-acetic acid (IAA), 0.1 mg/l gibberellic acid (GA₃), pH 5.6 and 8 WI Phytagar.

The explants are subjected to microprojectile bombardment prior to Agrobacterium treatment (Bidney, et al., (1992) Plant Mol. Biol. 18:301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA105 is used in all transformation experiments. A binary plasmid vector comprising the expression cassette that contains the urea transporter, urease, or glutamine synthetase gene operably linked to the ubiquitin promoter is introduced into Agrobacterium strain EHA105 via freeze-thawing as described by Holsters, et al., (1978) Mol. Gen. Genet. 163:181-187. This plasmid further comprises a kanamycin selectable marker gene (i.e, nptII). Bacteria for plant transformation experiments are grown overnight (28° C. and 100 RPM continuous agitation) in liquid YEP medium (10 gm/l yeast extract, 10 gm/l Bacto® peptone and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for a modulation in meristem development (i.e., an alteration of size and appearance of shoot and floral meristems).

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% Gelrite®, pH 5.6) and grown under conditions described for explant culture. The upper portion of the seedling is removed, a 1 cm vertical slice is made in the hypocotyl, and the transformed shoot inserted into the cut. The entire area is wrapped with Parafilm® to secure the shoot. Grafted plants can be transferred to soil following one week of in vitro culture. Grafts in soil are maintained under high humidity conditions followed by a slow acclimatization to the greenhouse environment. Transformed sectors of TO plants (parental generation) maturing in the greenhouse are identified by NPTII ELISA and/or by urea transporter, urease, or glutamine synthetase activity analysis of leaf extracts while transgenic seeds harvested from NPTII-positive TO plants are identified by urea transporter, urease, or glutamine synthetase activity analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox® bleach solution with the addition of two to three drops of Tween® 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA and 0.8% Phytagar at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar) and then cultured on the medium for 24 hours in the dark.

Approximately 18.8 mg of 1.8 μm tungsten particles are resuspended in 150 μl absolute ethanol. After sonication, 8 μl of it is dropped on the center of the surface of macrocarrier. Each plate is bombarded twice with 650 psi rupture discs in the first shelf at 26 mm of Hg helium gun vacuum.

The plasmid of interest is introduced into Agrobacterium tumefaciens strain EHA105 via freeze thawing as described previously. The pellet of overnight-grown bacteria at 28° C. in a liquid YEP medium (10 g/l yeast extract, 10 g/l Bacto® peptone and 5 g/l NaCl, pH 7.0) in the presence of 50 μg/l kanamycin is resuspended in an inoculation medium (12.5 mM 2-mM 2-(N-morpholino) ethanesulfonic acid, MES, 1 g/l NH₄Cl and 0.3 g/l MgSO₄ at pH 5.7) to reach a final concentration of 4.0 at OD₆₀₀. Particle-bombarded explants are transferred to GBA medium (374E) and a droplet of bacteria suspension is placed directly onto the top of the meristem. The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour day and 26° C. incubation conditions.

Explants (around 2 cm long) from two weeks of culture in 374C medium are screened for a modulation in meristem development (i.e., an alteration of size and appearance of shoot and floral meristems). After positive (i.e., an increase in urea transporter, urease or glutamine synthetase expression) explants are identified, those shoots that fail to exhibit an increase in urea transport, urea breakdown or ammonia assimilation activity are discarded and every positive explant is subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate protein activity assay. At this time, the positive shoots recovered from a single node will generally have been enriched in the transgenic sector detected in the initial assay prior to nodal culture.

Recovered shoots positive for an increased urea transporter, urease, or glutamine synthetase expression are grafted to Pioneer hybrid 6440 in vitro-grown sunflower seedling rootstock. The rootstocks are prepared in the following manner. Seeds are dehulled and surface-sterilized for 20 minutes in a 20% Clorox® bleach solution with the addition of two to three drops of Tween® 20 per 100 ml of solution and are rinsed three times with distilled water. The sterilized seeds are germinated on the filter moistened with water for three days, then they are transferred into 48 medium (half-strength MS salt, 0.5% sucrose, 0.3% Gelrite® pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with Parafilm®. After one week of culture on the medium, grafted plants are transferred to soil. In the first two weeks, they are maintained under high humidity conditions to acclimatize to a greenhouse environment.

### Example 14

### Variants of Urea Transporter, Urease, or Glutamine Synthetase Sequences

A. Variant Nucleotide Sequences of Urea Transporter, Urease or Glutamine

Synthetase that do not aLter the Encoded Amino Acid Sequence

The urea transporter, urease, or glutamine synthetase nucleotide sequences are used to generate variant nucleotide sequences having the nucleotide sequence of the open reading frame with about 70%, 75%, 80%, 85%, 90% and 95% nucleotide sequence identity when compared to the starting unaltered ORF nucleotide sequence of the corresponding SEQ ID NO. These functional variants are generated using a standard codon table. While the nucleotide sequence of the variants are altered, the amino acid sequence encoded by the open reading frames do not change.

B. Variant Amino Acid Sequences of Urea Transporter, Urease or Glutamine Synthetase Polypeptides

Variant amino acid sequences of the urea transporter, urease, or glutamine synthetase polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using the protein alignment set forth in FIG. 6 or 8, an appropriate amino acid can be changed. Once the targeted amino acid is identified, the procedure outlined in the following section C is followed. Variants having about 70%, 75%, 80%, 85%, 90% and 95% nucleic acid sequence identity are generated using this method.

C. Additional Variant Amino Acid Sequences of Urea Transporter, Urease or Glutamine Synthetase Polypeptides

In this example, artificial protein sequences are created having 80%, 85%, 90% and 95% identity relative to the reference protein sequence. This latter effort requires identifying conserved and variable regions from the alignment set forth in FIG. 6 or 8 and then the judicious application of an amino acid substitutions table. These parts will be discussed in more detail below.

Largely, the determination of which amino acid sequences are altered is made based on the conserved regions among urea transporter, urease or glutamine synthetase protein or among the other urea transporter, urease, or glutamine synthetase polypeptides. Based on the sequence alignment, the various regions of the urea transporter, urease, or glutamine synthetase polypeptide that can likely be altered are represented in lower case letters, while the conserved regions are represented by capital letters. It is recognized that conservative substitutions can be made in the conserved regions below without altering function. In addition, one of skill will understand that functional variants of the urea transporter, urease, or glutamine synthetase sequence of the invention can have minor non-conserved amino acid alterations in the conserved domain.

Artificial protein sequences are then created that are different from the original in the intervals of 80-85%, 85-90%, 90-95% and 95-100% identity. Midpoints of these intervals are targeted, with liberal latitude of plus or minus 1%, for example. The amino acids substitutions will be effected by a custom Perl script. The substitution table is provided below in Table 12.

TABLE 12
Substitution Table

| | Strongly | | |
|---|---|---|---|
| | Similar and | Rank of | |
| | Optimal | Order to | |
| Amino Acid | Substitution | Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C and P are not changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involve a 50:50 substitution of the two alternate optimal substitutions.

The variant amino acid sequences are written as output. Perl script is used to calculate the percent identities. Using this procedure, variants of the Dur3 and Urel polypeptides are generating having about 80%, 85%, 90% and 95% amino acid identity to the starting unaltered ORF nucleotide sequence.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

What is claimed is:

1. An isolated polynucleotide that modulates urea assimilation selected from the group consisting of:
a. a polynucleotide encoding a polypeptide having at least 80% sequence identity, as determined by the GAP algorithm under default parameters, to the full length sequence of a polypeptide selected from the group consisting of SEQ ID NOS: 224-250, 300, 302 and 321, wherein the polypeptide functions as a modifier of urea uptake;
b. a polynucleotide that is at least 80% identical as determined by the GAP algorithm to a polynucleotide sequence selected from the group consisting of SEQ ID NO: 224-250, 300, 302 and 321;
c. a polynucleotide selected from the group consisting of SEQ ID NOS: 224-250, 300, 302 and 321; and
d. a polynucleotide which is complementary to the polynucleotide of (a), (b) or (c).

2. A recombinant expression cassette, comprising the polynucleotide of claim 1, wherein the polynucleotide is operably linked, in sense orientation, to a promoter.

3. A host cell comprising the expression cassette of claim 2.

4. A transgenic plant comprising the recombinant expression cassette of claim 2.

5. The transgenic plant of claim 4, wherein said plant is a monocot.

6. The transgenic plant of claim 4, wherein said plant is a dicot.

7. The transgenic plant of claim 4, wherein said plant is selected from the group consisting of: maize, soybean, sunflower, sorghum, canola, wheat, alfalfa, cotton, rice, barley, millets, peanut, switchgrass, myscanthus, triticale and cocoa.

8. A transgenic seed from the transgenic plant of claim 4.

9. A method of modulating the urea transporter, urease or glutamine synthetase either alone or in combination in a plant, the method comprising:
a. introducing into a plant cell a recombinant expression cassette comprising the polynucleotide of claim 1 operably linked to a promoter;
b. culturing the plant cell under plant cell growing conditions; and
c. regenerating a plant form said plant cell; wherein the urea transporter, urease, or glutamine synthetase in said plant is modulated.

10. The method of claim 9, wherein the plant is selected from the group consisting of: maize, soybean, alfalfa, barley, canola, cocoa, cotton, millets, myscanthus, peanut, rice, rye, sorghum, sugar cane, switchgrass, triticale and wheat.

11. A method of increasing the urea assimilation activity in a plant cell, comprising:
a. providing a nucleotide sequence encoding a urea transporter having at least 90% sequence identity to SEQ ID NO: 1-223, 251-299, 301 and 322;
b. linking the nucleotide to one or more nucleotide sequences selected from the group consisting of: an additional urea transporter, a urease and a glutamine synthase; and
c. introducing the nucleotide sequence of step (a) and the optionally linked sequences of (b) into the plant cell, wherein urea assimilation in the plant cell is increased.

12. The method of claim 9, wherein said plant cell is from a monocot.

13. The method of claim 12, wherein said monocot is maize, soybean, alfalfa, barley, canola, cocoa, cotton, millets, myscanthus, peanut, rice, rye, sorghum, sugar cane, switchgrass, triticale or wheat

14. The method of claim 9, wherein said plant cell is from a dicot.

15. The transgenic plant of claim 4, wherein the urea transport, urea breakdown, or ammonia assimilation activity in said plant is increased.

16. The transgenic plant of claim 15, wherein the plant has increased seedling vigor.

17. The transgenic plant of claim 15, wherein the plant has enhanced silk emergence.

18. The transgenic plant of claim 15, wherein the plant has enhanced nitrogen assimilation in roots.

19. The transgenic plant of claim 15, wherein the plant has increased seed number per plant.

20. The transgenic plant of claim 15, wherein the plant has increased seed size and mass.

21. The transgenic plant of claim 15, wherein the plant has seed with increased embryo size.

22. The transgenic plant of claim 15, wherein the plant has increased nitrogen assimilation in the leaf.

23. The transgenic plant of claim 15, wherein the plant has increased ear size.

24. The transgenic plant of claim 15, wherein the plant has enhanced nitrogen utilization efficiency.

25. The transgenic plant of claim 15, wherein the plant has increased nitrogen remobilization during senescence.

26. The transgenic plant of claim 15, wherein the plant has increased nitrogen remobilization during grain development.

27. A method of modifying urea uptake comprising:
a. transforming a corn plant with a high affinity urea transporter and a low affinity urea transporter; and
b. expressing the low affinity and high affinity urea transporters whereby urea uptake the transgenic plant is improved as compared to a control plant.

28. The method of claim 27, wherein the low affinity and the high affinity urea transporters are active at different growth stages of the corn.

29. The method of claim 27, wherein the low affinity and the high affinity urea transporters are active depending on the available urea concentrations in the soil.

30. The method of claim 27 wherein the transporters are Dur3 or Urel family members

31. The method of claim 27 wherein the plant is transformed with an expression cassette comprising a promoter selected from the group comprising a urea regulated promoter, a constitutive or a root preferred promoter.

32. A method of increasing yield by applying a nitrogen source to a plant at or during the reproductive stage, the method comprising:
(a) growing the plant to a reproductive stage; and
(b) providing a foliar application of urea to the plant, wherein the plant is capable of utilizing the foliar application of urea through the expression of one or more urea transporters in the leaf, whereby the yield of the plant is increased.

33. A method of reducing nitrogen loss from an agricultural production environment, the method comprising:
(a) growing a plant that is capable of utilizing urea as a direct nitrogen source from the soil or through a foliar application;
(b) providing an urea containing nutrient source to the plant;
(c) growing the plant and thereby reducing the loss of nitrogen from the agricultural production environment.

34. The method of claim 33, wherein the plant expresses an urea transporter.

35. The method of claim 34, wherein the urea transporter is a high affinity transporter.

36. The method of claim 33, wherein the loss of nitrogen is reduced through an increased uptake of urea by the plant.
